(19) United States
(12) Patent Application Publication
(10) Pub. No.: US 2024/0226105 A1
(43) Pub. Date: Jul. 11, 2024

(54) TETRODOTOXIN LIQUID FORMULATIONS

(71) Applicant: Wex Pharmaceuticals Inc., Vancouver (CA)

(72) Inventors: Meng ZHOU, Oakville (CA); Donald C. K. WONG, Vancouver (CA)

(73) Assignee: Wex Pharmaceuticals Inc., Vancouver, BC (CA)

(21) Appl. No.: 18/556,683

(22) PCT Filed: Apr. 22, 2022
(86) PCT No.: PCT/CA2022/050623

§ 371 Date: Oct. 23, 2023

Related U.S. Application Data
(60) Provisional application No. 63/201,331, filed on Apr. 23, 2021.

(51) Int. Cl. A61K 31/519; A61K 9/00; A61K 9/19; A61K 47/02; A61K 47/10; A61K 47/12; A61K 47/18; A61K 47/20

(52) U.S. Cl. CPC A61K 31/519; A61K 9/0019; A61K 9/19; A61K 47/02; A61K 47/10; A61K 47/12; A61K 47/18; A61K 47/20

(57) ABSTRACT

A stable formulation comprising tetrodotoxin, and/or a derivative, analog, or a pharmaceutically acceptable salt thereof, wherein the formulation comprises the tetrodotoxin component and one or more solvents, pH adjusting agents, buffering agents, and stabilizing agents.

### FIELD OF THE DESCRIPTION

The present description relates generally to liquid formulations comprising tetrodotoxin. In one aspect, the description relates to stable tetrodotoxin formulations that are suitable for use as pharmaceutical product. Such products may be in a form for injection to a mammal.

### BACKGROUND

Tetrodotoxin is a naturally occurring, non-protein marine neurotoxin. Tetrodotoxin binds with the SS1/SS2 subunit of voltage-gated sodium channels with high specificity and high affinity.

The chemical name of tetrodotoxin (C₁₁H₁₇N₃O₈) is octahydro-12-(hydroxymethyl)-2-imino-5,9:7,10a-dimethano-10aH-(1,3)dioxocino(6,5-d)-pyrimidine-4,7,10,11,12-pentanol. Its molecular weight 319.28, and has the following structure:

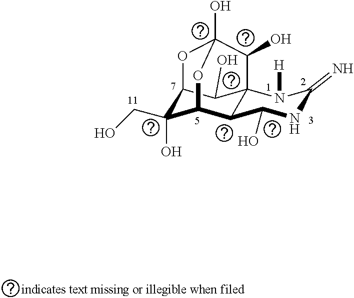

Tetrodotoxin (or “TTX”) is a white to off-white crystalline powder that darkens above 220° C. without decomposition and has the following characteristics: [a]D25-8.64 (C=8.55 in diluted acetic acid); pKa 8.76 in water and 9.4 in 50% alcohol. TTX is water soluble and generally insoluble in organic solvents. TTX is relatively stable at acidic pH, but quickly degrades in alkaline solution.

Most likely due to its being a strong neurotoxin, TTX has not been extensively investigated. Nevertheless, tetrodotoxin has been known to be used as a tool in pharmacological research, particularly neurophysiology and ion-channel electrophysiology. In addition to its use in scientific research, therapeutic applications of TTX have also been described. In some cases, a tetrodotoxin compositions for injection (i.e., in the form of aqueous solutions), have been described. For example, TTX and derivatives thereof have been described for use as non-opioid analgesics in the treatment of chemotherapy induced neuropathic pain (CINP) and cancer related pain (CRP). These and other examples of uses of TTX have been described in, for example, U.S. Pat. Nos. 5,846,975; 6,407,088; 6,599,906; 8,486,901; 9,018,222; and 10,624,896.

One of the problems encountered in using TTX as a therapeutic has been its tendency to rapidly degrade when in liquid solution form, in particular as a result of the molecule's high sensitivity to temperature. In U.S. Pat. No. 8,124,608, it was found that the content of tetrodotoxin, as examined by HPLC, declined to 91.9% on day 1 and further to 89.37% on day 3, when stored in aqueous solution form at 40° C. Moreover, the content of tetrodotoxin declined to 95.34% when the formulation was left standing for one month at 25° C., and 89.77% after three months at 25° C. As will be understood, once the content of tetrodotoxin, the active pharmaceutical ingredient in such formulations, is reduced to less than 90% of the labelled amount, the formulation is no longer suitable for clinical use.

Thus, in view of the lack of acceptable stability, and consequently unacceptable shelf life, the currently known TTX formulations are not suitable for wide-scale application in treatment methods.

The characterization of the tetrodotoxin degradation products and the mechanism of tetrodotoxin degradation in aqueous media have been extensively studied. In 1965, T. Goto et al. reported that tetrodotoxin transformed to an epimer, 4-epi-tetrodotoxin under acidic medium. The epimer can further dehydrate to form 4,9-anydro-tetrodotoxin (Tetrahedron. 1965, Vol 21, 2059-2088). The epimerization of tetrodotoxin is facilitated by a proton via a rapid ring-opening intermediate:

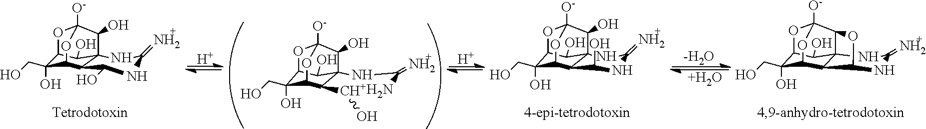

Upon mild water treatment of tetrodotoxin, T. Goto et al., found the formation of tetrodonic acid (Tetrahedron Lett., 1963, 4, 2105-2113), as follows:

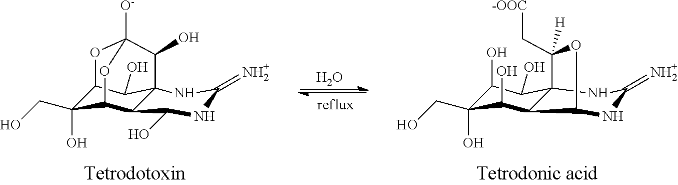

At extreme acidic or alkaline condition, tetrodotoxin breaks down into other complex degradation products (Woodward, R. B. The structure of tetrodotoxin. Pure and Appl. Chemistry, 1964, 9(1), 49-74.)

To address the problem associated with the degradation of tetrodotoxin in aqueous solution, a lyophilized tetrodotoxin powder formulation has been proposed, such as described in U.S. Pat. Nos. 8,124,608; 8,222,258; and 8,530,481. However, lyophilized powder formulations has several disadvantages. First, the lyophilization process incurs a significant manufacturing cost per dose when considered in an industrial scale, which thereby leads to increased costs for the therapeutic products. Second, use of a conventional lyophilized formulation, which is typically in a powdered form, first requires reconstitution in a diluent, such as water for injection (WFI), or 0.9% sodium chloride injection (USP), in order for the drug to be administered. Reconstitution is an aseptic technique that generally requires the skill of a healthcare professional and often a qualified pharmacy compounding facility. Third, as discussed above, TTX is soluble in water and not to any desired degree in organic solvents; however, as also discussed above, exposure to water accelerates the degradation of TTX. Consequently, formulations formed by reconstituting TTX in an aqueous solution are not sufficiently stable unless stored under special conditions.

There is, therefore, a need for a liquid tetrodotoxin formulation that addresses at least one of the aforementioned deficiencies.

### SUMMARY OF THE DESCRIPTION

In one aspect, there is provided a stable liquid formulation comprising tetrodotoxin, and/or a derivative, analog, or a pharmaceutically acceptable salt thereof, and one or more pharmaceutically acceptable diluents, carriers, and excipients.

In one aspect, the formulation comprises less than 15% water (w/v). In another aspect, the formulation comprises up to 95% polyethylene glycol (PEG) (w/v).

In one aspect, the stable formulation is provided in a container. In another aspect, the stable formulation is provided in a pre-filled syringe adapted for subcutaneous administration of tetrodotoxin. In another aspect, the formulation is adapted to administer a tetrodotoxin dose of 5-120, or 15-60, or about 30 μg to a subject.

In one aspect, there is provided a use of the stable liquid formulation comprising TTX for the treatment of pain. In a further aspect the pain is chemotherapy induced neuropathic pain (CINP).

In another aspect, there is provided the stable liquid formulation comprising TTX for use in the treatment pain. In a further aspect the pain is chemotherapy induced neuropathic pain (CINP).

In another aspect, there is provided a method of administering the stable liquid formulation comprising TTX to a patient in need thereof for the treatment of pain. In a further aspect the pain is chemotherapy induced neuropathic pain (CINP).

### BRIEF DESCRIPTION OF FIGURES The features of certain embodiments will become more apparent in the following detailed description in which reference is made to the appended figures wherein:

FIG. 1 is a graph of TTX stability vs. PG/PEG 400 ratio at 2 and 4 weeks at 40° C./75% RH. The graph illustrates tetrodotoxin (“TTX”) stability in formulations comprising different ratios of propylene glycol (“PG”) and polyethylene glycol 400 (PEG 400) as an organic solvent.

FIG. 2 is a graph of TTX stability vs. pH at 2 and 4 weeks at 40° C./75% RH. The graph illustrates TTX stability in formulations having different pH and with an acid/PG/PEG 400 ratio of 5/50/45 (v/v).

FIG. 3 is a graph of TTX stability vs. pH at 2 and 4 weeks at 40° C./75% RH. The graph illustrates TTX stability in formulations having different pH and with an acid/PG/PEG 400 ratio of 5/80/15 (v/v).

FIG. 4 is a graph illustrating the effects of two TTX formulations on pain withdrawal threshold (PWT) in oxaliplatin induced pain model rats. Markings on the graph indicate the following: *, **, ***: p<0.05, 0.01, 0.001, respectively, compared to G1 Saline group, one-way ANOVA, n=7.

FIG. 5 is a graph illustrating the effects of two TTX formulations on PWT in oxaliplatin induced pain model in rats at one hour post dose. Markings on the graph indicate the following: *, **, ***: p<0.05, 0.01, 0.001, respectively, compared to G1 Saline group, one-way ANOVA, n=7.

FIG. 6 is a graph illustrating the effects of two TTX formulations on PWT in oxaliplatin induced pain model in rats at 2 hours post dose. Markings on the graph indicate the following: *, **, ***: p<0.05, 0.01, 0.001, respectively, compared to G1 Saline group, one-way ANOVA, n=7.

FIG. 7 is a graph illustrating the effect of two TTX formulations on baseline PWT in oxaliplatin induced pain model in rats. Markings on the graph indicate the following: *, **, ***: p<0.05, 0.01, 0.001, respectively, compared to G1 Saline group, one-way ANOVA, n=7.

### DETAILED DESCRIPTION

As described herein, there are provided liquid pharmaceutical formulations of tetrodotoxin, or derivatives or analogs thereof, or pharmaceutically acceptable salts thereof. As will be apparent from the present description, the tetrodotoxin liquid formulations disclosed herein have several advantages over known lyophilized formulations. For example, the presently described tetrodotoxin liquid formulations do not require a lyophilization process, which results in reduced manufacturing costs, which is particularly of importance when manufacturing doses on an industrial scale. The described liquid formulations do not need to be reconstituted, thereby avoiding the expense associated therewith and the need for health care practitioners required for such reconstitution. As such, the formulations described herein may be provided or packaged in a container, such as a syringe, an ampule, a vial, or an autoinjector, etc. Such containers may be made of glass, plastic, or another other material as known in the art. In one aspect, the described tetrodotoxin liquid formulations can be provided in a “ready to use” form for self-administration by a patient, or for administration by any other person, using an injection device. In this regard, the presently described formulation may be provided in the form of pre-filled syringes, or other known administration devices, having a pre-set dosage of tetrodotoxin. As would be understood, in using the described liquid formulations, administration errors may also be reduced since the formulations do not require reconstitution. As would also be understood, such administration errors can be highly detrimental to the patient owing to the fact that tetrodotoxin is a highly toxic substance.

### Definitions

Unless stated otherwise herein, the articles “a” or “the”, when used to identify an element, are not intended to constitute a limitation of just one and will, instead, be understood to mean “at least one” or “one or more”. Thus, unless stated otherwise, as used in this specification and the appended claims, the singular forms “a”, “an”, and “the” will be understood to include the plural form. For example, reference to “a container” will be understood to include one or more of such containers and reference to “the excipient” will be understood to include one or more of such excipients.

As used herein, the term “tetrodotoxin” refers to a naturally occurring, synthetic, or semi-synthetic chemical with the general formula provided above, and pharmaceutically acceptable salts thereof.

As used herein, the terms “derivatives of tetrodotoxin” and “analogs of tetrodotoxin” refer, but are not limited to anhydro-tetrodotoxin, tetrodaminotoxin, methoxytetrodotoxin, ethoxytetrodotoxin, deoxytetrodotoxin, tetrodonic acid, 6-epi-tetrodotoxin, 11-doexytetrodotoxin as well as the hemilactal type tetrodotoxin analogs (e.g. 4-epi-tetrodotoxin, 6-epi-tetrodotoxin, 1′-deoxy-tetrodotoxin, 4-epi-11-deoxy-tetrodotoxin, tetrodotoxin-8-O-hemisuccinate, chiriquitoxin, 11-nor-tetrodotoxin-6(S)-ol, 1′-nor-tetrodotoxin-6(R)-ol, 11-nor-tetrodotoxin-6,6-diol, 11-oxo-tetrodotoxin, and tetrodotoxin-11-carboxylic acid), the lactone type tetrodotoxin analogs (e.g.6-epi-tetrodotoxin (lactone), 11-deoxy-tetrodotoxin (lactone), 1′-nor-tetrodotoxin-6(S)-ol (lactone), 11-nor-tetrodotoxin-6(R)-ol (lactone), 11-nor-tetrodotoxin-6,6-diol (lactone), 5-deoxy-tetrodotoxin, 5,11-dideoxy-tetrodotoxin, 4-epi-5,11-dideoxy-tetrodotoxin, 1-hydroxy-5,11-dideoxy-tetrodotoxin, 5,6,11-trideoxy-tetrodotoxin and 4-epi-5,6,11-trideoxy-tetrodotoxin), and the 4,9-anhydro type tetrodotoxin analogs (e.g. 4,9-anhydro-tetrodotoxin, 4,9-anhydro-6-epi-tetrodotoxin, 4,9-anhydro-11-deoxy-tetrodotoxin, 4,9-anhydro-tetrodotoxin-8-O-hemisuccinate, 4,9-anhydro-tetrodotoxin-11-O-hemisuccinate), and pharmaceutically acceptable salts thereof.

Unless stated otherwise herein, references to “tetrodotoxin” or “TTX” will be understood to include the naturally occurring substance or derivatives and/or analogs thereof.

As used herein, the term “subject” or “patient” refers to a mammal. Examples of subjects include humans, and may also include other animals such as horses, pigs, cattle, dogs, cats, rats, rabbits, and aquatic mammals. The present description is not limited to any particular mammal; however, it will be understood that the preferred subjects are humans.

As used herein, “treat”, “treating”, or “treatment” means the treatment of a disease in a subject, for example a human, and includes inhibiting the disease (e.g., decreasing its rate of progression); regressing the disease; relieving or decreasing the severity of one or more symptoms of the disease; and/or curing the disease.

As used herein, “prevent,” “preventing”, or “prevention” means the prevention of a disease in a subject and includes inhibiting initiation of the disease; decreasing a predisposition toward the disease; and/or delaying the onset of at least one symptom of the disease.

As used herein, the term “about” is synonymous with “approximately” and is used to provide flexibility to a numerical value, or to the start- and endpoints of range, by providing that a given value may be “a little above” or “a little below” the value stated. “About” can mean, for example, within three or more than three standard deviations. “About” can mean within a percentage range of a given value. For example, the range can be ±1%, ±5%, ±10%, ±20%, ±30%, ±40% or ±50% of a given value. “About” can mean with an order of magnitude of a given value, for example, within 2-fold, 3-fold, 4-fold, or 5-fold of a value. However, it is to be understood that even when a numerical value is characterized herein by the term “about”, express support shall be provided at least for the exact numerical value as though the term “about” were not present. In one aspect, the term about will be understood to encompass a range ±10% of the respective value.

The term “and/or” can mean “and” or “or”.

The terms “comprise”, “comprises”, “comprised”, or “comprising” may be used in the present description. As used herein (including the specification and/or the claims), these terms are to be interpreted as specifying the presence of the stated features, integers, steps, or components, but not as precluding the presence of one or more other feature, integer, step, component, or a group thereof as would be apparent to persons having ordinary skill in the relevant art. Thus, the term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

As used herein, “comprises,” “comprising,” “containing” and “having” and the like can have the meaning generally ascribed to them and can mean “includes”, “including”, and the like, and are generally interpreted to be open ended terms. The terms “consisting of” or “consists of” are closed terms, and include only the components, structures, steps, or the like specifically listed in conjunction with such terms.

The phrase “consisting essentially of” or “consists essentially of” will be understood as generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term, such as “comprising” or “including”, it will be understood that direct support should be afforded also to “consisting essentially of” language as well as “consisting of” language as if stated explicitly and vice versa. In essence, use of one of these terms in the specification provides support for all of the others.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but to also include all the individual numerical values or sub-ranges encompassed within that range as if each numerical value and sub-range is explicitly recited. As an illustration, a numerical range of “about 1 to about 5” should be interpreted to include not only the explicitly recited values of about (e.g., ±10%) 1 to about (e.g., ±10%) 5, but to also include individual values and sub-ranges within the indicated range. Thus, included in this numerical range are individual values such as about 2, about 3, and about 4 and sub-ranges such as from about 1 to about 3, from about 2 to about 4, and from about 3 to about 5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Stability of Tetrodotoxin Liquid Formulations

As used herein, the term “stable”, in the context of the present TTX formulations, will be understood to apply to a formulation wherein the TTX component does not degrade beyond a given value over a given period of time. Some of the known mechanisms of TTX degradation have been described above. In one aspect, a liquid TTX formulation may be described as being stable if the TTX component of the formulation does not degrade to greater degree than TTX in liquid formulations known in the art, examples of which are provided above. This stability criteria are particularly advantageous when the TTX liquid formulations are to be stored for immediate administration or self-administration to a subject in need thereof.

In one aspect, the stability of TTX can be evaluated or quantified by measuring the level or concentration of TTX in a formulation over a period of time and at predetermined conditions (e.g., at a specified temperature and relative humidity) and comparing such observations or measurements against known TTX formulations. In another aspect, the stability of a TTX formulation can be evaluated or quantified by measuring the presence, level, or concentration of one or more TTX degradation products in the formulation over a given period of time and under specified conditions. Thus, the stability of TTX formulations may be measured qualitatively or quantitatively by measuring the presence of degradation products and comparing such measurements to predetermined threshold values.

In one aspect, a stable TTX formulation may be defined as a formulation that is sufficiently effective (i.e., achieving the desired TTX response), and has a concentration of one or more TTX degradation products that is below a predetermined threshold. Sufficiently effective may be, for example, about 90%, 91%, 92%, 94%, 94%, 95%, 96%, 97%, 98%, 99%, or 100% effective in achieving the desired TTX response.

For example, in one aspect, a stable liquid TTX formulation may comprise a formulation of TTX wherein the TTX component does not degrade by more than 11% after being stored at 25° C. for three months. In another aspect, a stable TTX formulation may comprise a formulation of TTX wherein the TTX component does not degrade more than 5% after being stored at 25° C. for one month. In one aspect, the present description provides a TTX formulation that is (a) stable for 24 months at a temperature of 2-8° C.; and/or (b) stable for 1 month at 25° C.; and/or (c) stable for 28 days at a temperature of 40° C.

Thus, in one aspect, a stable TTX formulation according to the present description exhibits less than 5%, 10%, 15%, 20%, 25%, 30% or 35% degradation of the tetrodotoxin after the formulation is stored at 40° C. and 75% relative humidity (RH) for a period of 28 days. In one aspect, the formulation is defined as being stable if less than 10% of the tetrodotoxin undergoes degradation after being stored at 40° C. for a period of 28 days.

In another aspect, a stable TTX formulation according to the present description exhibits less than 5%, 10%, 15%, 20%, 25%, 30% or 35% degradation of the tetrodotoxin when the formulation is stored at 25° C. and 40% RH for a period of 12 weeks. In one aspect, the formulation is defined as stable if less than 10% of the tetrodotoxin undergoes degradation after being stored at 25° C. and 40% RH for a period of 12 weeks.

In another aspect, a stable TTX formulation according to the present description exhibits less than 5%, 10%, 15%, 20%, 25%, 30% or 35% degradation of the tetrodotoxin when the formulation is stored at 2-8° C. and ambient RH for a period of 6 months. In one aspect, the formulation is defined as stable if less than 5% of the tetrodotoxin undergoes degradation after being stored at 2-8° C. and ambient RH for a period of 6 months.

For the purposes of the present analysis, an analytical method was designed and validated for the quantification of TTX and TTX degradation products in TTX formulations. This method was applied to assay the degradation products mentioned above, namely, 4-epi-tetrodotoxin and 4,9-anydro-tetrodotoxin. Thus, an indication of stability of a TTX formulation may be quantified by measuring the concentration of one or both of these degradation products. On the basis of assays conducted, and purely by way of example, a TTX formulation may be deemed to be stable if over a predetermined period of time, the concentration of 4-epi-tetrodotoxin does not exceed about 2% and/or the concentration of 4,9-anydro-tetrodotoxin does not exceed about 5% in the formulation. It will be understood that the time for these degradation products to reach such concentrations will depend on the conditions to which the formulations are exposed. For example, it is understood that TTX degradation is accelerated at higher temperatures.

### Tetrodotoxin Liquid Formulations

Described herein are liquid formulations of tetrodotoxin, or derivatives or analogs thereof, or pharmaceutically acceptable salts thereof, that have been shown to have enhanced stability characteristics over known TTX liquid formulations, such as known formulations made by reconstituting lyophilized TTX. The term “stable” has been defined above and will be understood to mean that the TTX component in the formulation is not degraded by a given amount, and/or that the concentration of one or more TTX degradation products is not greater than a given amount.

The formulations described herein may be formulated in an injectable form and/or in the form of ready to administer compositions. Thus, contemplated herein are dosage forms that comprise, for example, pre-filled syringes or pre-filled ampules comprising a set dose of TTX for administration to a subject.

The liquid TTX formulations described herein comprise tetrodotoxin, water, a solvent, and one or more other components as discussed herein. In one aspect, the formulations may further comprise one or more pharmaceutically acceptable diluents, carriers, excipients etc. Preferably, the tetrodotoxin liquid formulations described herein contain a minimal amount of water that balances the solubility and stability of tetrodotoxin. In other words, in one aspect, the formulations described herein comprise a sufficient amount of water to solubilize the TTX component but not enough water to detrimentally affect the stability of such component.

In one embodiment, the liquid TTX formulation is adapted for parenteral administration and comprises:
(i) Tetrodotoxin, or a derivative of tetrodotoxin, or an analog of tetrodotoxin, or a pharmaceutically acceptable salt thereof;
(ii) water;
(iii) one or more pharmaceutically acceptable solvents;
(iv) one or more pharmaceutically acceptable pH adjusting agents;
(v) one or more pharmaceutically acceptable buffering agents; and
(vi) one or more pharmaceutically acceptable stabilizing agents.

Solvents suitable for use in the presently described formulation include, but are not limited to, ethyl alcohol (ethanol) (“EtOH”), dehydrated ethyl alcohol, denatured ethyl alcohol, benzyl alcohol, dimethyl sulfoxide, glycerin, isopropyl alcohol, methylpyrrolidone, N,N-dimethylacetamide (“DMA”), polyethylene glycol 200 (“PEG 200”), polyethylene glycol 300 (“PEG 300”), polyethylene glycol 400 (“PEG 400”), polyethylene glycol 600 (“PEG 600”), polypropylene glycol, propylene glycol (“PG”), diethylene glycol monoethyl ether, or any combination thereof.

The pH adjusting agents suitable for use in the presently described formulation include, but are not limited to, hydrochloric acid, acetic acid (“AA”), acetic anhydride, adipic acid, anhydrous citric acid, benzenesulfonic acid, boric acid, citric acid monohydrate, lactic acid, (DL)-lactic acid, (L)-lactic acid, maleic acid, metaphosphoric acid, methanesulfonic acid (“MSA”), nitric acid, phosphoric acid, succinic acid, sulfuric acid, sulfurous acid, tartaric acid, (DL)-tartaric acid, trifluoroacetic acid, ascorbic acid, benzoic acid, edetic acid, formic acid, lactobionic acid, aspartic acid, caprylic acid, glucuronic acid, hydroxyethylpiperazine ethane sulfonic acid, methylboronic acid, oleic acid, palmitic acid, pentetic acid, stearic acid, sodium hydroxide, calcium hydroxide, potassium hydroxide, sodium bicarbonate, sodium carbonate, sodium carbonate decahydrate, sodium carbonate monohydrate, diethanolamine, meglumine, tromethamine, ammonia, or any combination thereof.

Buffering agents used in the presently described formulation include, but are not limited to sodium phosphate, dibasic, heptahydrated sodium phosphate, dibasic, sodium phosphate, dibasic, anhydrous, sodium phosphate, dibasic dehydrate, sodium phosphate, dibasic dodecahydrate, sodium phosphate, sodium phosphate dehydrate, sodium phosphate, monobasic, anhydrous, sodium phosphate, monobasic, dehydrate, sodium phosphate, monobasic, monohydrate, dibasic potassium phosphate, potassium phosphate, monobasic, sodium acetate, sodium acetate anhydrous, ammonium acetate, sodium citrate, disodium hydrogen citrate, anhydrous trisodium citrate, disodium citrate sesquihydrate, trisodium citrate dehydrate, sodium lactate, (L)-sodium lactate, sodium tartrate, ammonium sulfate, ethanolamine hydrochloride, or any combination thereof.

Stabilizing agents used in the presently described formulation may comprise one or more known pharmaceutically acceptable antioxidants, surfactants, preservatives, sugars. Such agents include, but are not limited to sodium sulfite, sodium bisulfite, sodium metabisulfite, potassium metabisulfite, alpha-tocopherol, acetone sodium bisulfite, ascorbic acid, sodium ascorbate, butylated hydroxyanisole, butylated hydroxytoluene, gentisic acid, gentisic ethanolamide, glutathione, methionine, monothioglycerol, sodium formaldehyde sulfoxylate, edetate disodium, edetate disodium anhydrous, edetate sodium, edetate calcium disodium, edetate calcium disodium anhydrous, edetic acid, gluceptate sodium, pentasodium pentetate, pentetate calcium trisodium, pentetic acid, povidone, povidone K12, povidone K17, crospovidone, carboxymethylcellulose, methylcellulose, microcrystalline cellulose, poloxamer 188, polyvinyl alcohol, polysorbate 80, sorbitan monolaurate, sorbitan monopalmitate, hydroxypropyl betacyclodextrin, gamma cyclodextrin, sulfobutylether betacyclodextrin (“SBECD”), sorbitol, sorbitol solution, lactose monohydrate, mannitol, fructose, gluconolactone, sucrose, trehalose, guanidine, guanidine hydrochloride, benzalkonium chloride, phenylethyl alcohol, propylparaben, methylparaben, hexylresorcinol, succimer, butylparaben, metacresol, miripirium chloride, phenol, propyl gallate, or any combination thereof.

In one aspect, the formulation described herein is packaged in a container. In one aspect, the container may comprise a syringe, an ampule, a vial, or an autoinjector. In one aspect, the formulation may be provided in such container in a predetermined volume and/or TTX concentration, thereby resulting in a predetermined dose of TTX for administration.

In another aspect, the container may be provided in a kit comprising the container and a suitable administration device, where needed. For example, the kit may comprise an ampule or vial containing a volume of the formulation and a syringe for administering the formulation to a subject. The kit may also comprise suitable instructions and other information and/or equipment to aid in the administration or self-administration of the TTX formulation.

The subject formulations can be prepared using techniques described herein. For example, the formulations may be prepared by first dissolving TTX (or a derivative, analog, or salt thereof) in water. One or more pH adjusting agents, and/or one or more buffering agents, may also be combined in this aqueous solution. One or more stabilizing agents may then be combined, followed by mixing one or more solvents. The composition can be aseptically filled into a container suitable for direct administration (i.e., without requiring any further mixing, diluting, reconstituting, etc.)

The studies summarized herein illustrate the ability of providing stable TTX liquid formulations. The formulations can be provided in containers and stored in liquid form for a sufficient period of time, without degradation, for later use. Thus, the formulations provided herein are believed to be the first commercially viable liquid TTX formulations that are stable. The formulation can therefore be provided in predetermined doses for administration by, for example, sub-cutaneous (“S.C.”) or intramuscular (“I.M.”) injection. In this regard, the TTX formulation can be provided in a pre-filled syringe. In this way, the formulation can, in one aspect, be provided in a form for self-administration.

It will be understood that the TTX formulations described herein may be provided in any suitable concentration and/or dose amount. As will be understood, the required TTX dosage and concentration are closely related to the volume of the dose to be delivered.

In one example, the TTX formulations described herein may be provided with a TTX concentration from about 5 to 5000 μg/ml. Included in this range are concentrations of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, 550, 600, 650, 700, 750, 800, 850, 900, 950, 1000, 1500, 2000, 2500, 3000, 3500, 4000, 4500, and 5000 μg/ml, including dosage amounts between these values.

In one example, the formulation described herein may comprise a TTX dose of from about 5 to about 120 μg. In another example, the formulation described herein may comprise a TTX dose from about 15 to about 60 μg. In another example, the formulation described herein may comprise a TTX dose of about 30 μg.

As will be understood, the required TTX dosage and concentration are closely related to the volume of the dose to be delivered. In one example, the formulation may comprise from about 60 μg/ml of TTX, thereby allowing for a dose of 30 μg TTX with about 0.5 ml of the formulation, which is suitable for S.C. administration. In another example, the formulation may comprise from about 100 μg/ml of TTX, thereby allowing for a dose of 30 μg TTX with about 0.3 ml of the formulation, which is suitable for S.C. administration. In another example, the formulation may comprise from about 150 μg/ml of TTX, thereby allowing for a dose of 30 μg TTX with about 0.2 ml of the formulation, which is suitable for S.C. administration.

### EXAMPLES

The examples provided herein are included solely for the purpose of illustration and are not intended to limit the scope of any invention described herein. The examples serve to illustrate specific aspects of the description. Persons skilled in the art will appreciate that the scope of the present description will comprise additional aspects that can be reasonably predicted based on the factual bases provided herein.

The aim of the examples was to arrive at a liquid TTX formulation that would provide a fixed TTX dose of up to 30 μg. For this reason, the aim was to arrive at a formulation having a TTX concentration of 300 μg/ml, which would allow for a S.C. or I.M. administration of roughly 0.1 to 0.2 ml of the formulation.

As discussed above, TTX is known to be heat sensitive; e.g., TTX rapidly degrades at 40° C. Thus, it was assumed that using 40° C. as a study condition would provide better screening when assessing stability of the formulations. In other words, exposing the formulations to conditions of 40° C. for 1 month was assumed to be equivalent to a 24-month study at 5° C.

As also discussed above, TTX is known to have better stability characteristics in acidic environments. In particular, TTX is labile in alkaline, pH>6, so the present study sought to arrive at a preferred formulation pH of 3-5. In some of the tests, a pH of 4 was used for the analyses.

As is also known, TTX is water soluble but is also quickly degraded in such aqueous environment. The present study therefore sought to determine what amount of water provides an optimal balance of the need for water to enhance the dissolution of TTX with the degradation of TTX resulting from exposure to water.

### A) Solubility Studies

### Example 1—Solubility of TTX in Organic Solvent and Acid

In this study, the solubility of TTX in various acid and organic solvent combinations was investigated to determine solubility characteristics of TTX in an environment essentially free of water. The target TTX concentration was 30 μg/mL. The acids studied were ascorbic acid (“AA”), citric acid (“CA”), and methanesulfonic acid (“MSA”). The solvents studied were ethanol (“EtOH”) and benzyl alcohol (“BA”). The results of this study are presented below in Table 1.

TABLE 1

| | Acid | | | Initial | | TTX |
|---|---|---|---|---|---|---|
| | Solubility | Conc. | | pH | Dilution/pH | Solubility |
| AA in EtOH | Readily | 0.4% | w/v | 3.5 | 1^{st} Dilution: 50/50 | Not soluble |
| | soluble | | | | (AA/EtOH); pH = 3.7 | at 30 μg/mL |
| | | | | | 2^{nd} Dilution: 20/80; | |
| | | | | | pH = 3.9; | |
| | | | | | 3^{rd} Dilution 10/90; | |
| | | | | | pH = 4.0 | |
| MSA in EtOH | Fully | 8.07% | w/v | Negative | Dilution: 50/50; | |
| | solubilized | | | pH | Negative pH | |
| CA in EtOH | Not soluble | | | | | |
| AA in BA | Readily | 0.4% | w/v | 4.6 | Not needed | Not soluble |
| | soluble | | | | | at 30 μg/mL |
| MSA in BA | Lump formed | 8.07% | w/v | Negative | Dilution: 50/50; | |

| | | | |
|---|---|---|---|
| | first, but | pH | Negative pH |
| | solubilized | | |
| CA in BA | Not soluble | | |

As noted, TTX was found to be insoluble in AA/EtOH and AA/BA mixtures. MSA in EtOH was unusable in view of the extremely low pH. CA was found to be insoluble in either EtOH or BA, so a mixture for TTX dissolution could not be formed. These findings suggest some incompatibility between some acids and solvents.

The above findings are not surprising owing to the fact that TTX is a zwitterion in aqueous solution and, therefore, organic solvents are unsuitable media to facilitate TTX ionization and protonation. The results from the studies conducted demonstrates, as expected, that water is required to achieve a TTX solution.

### Example 2—Solubility of TTX in Water and Acid

In this series of tests an investigation was conducted of the solubility of TTX in an aqueous acid solution, i.e., without an organic phase. The purpose of this investigation was to also assess the effect of pH on the solubility of TTX in water. For this purpose, TTX was added to aqueous solutions of acetic acid (“AA”) at concentrations of 1.0, 1.5, 2.0, and 3.0 mg/mL. The results of these solubility studies are presented in Table 2.

TABLE 2

| AA | TTX concentration | | | |
|---|---|---|---|---|
| solution | 1.0 mg/mL | 1.5 mg/mL | 2.0 mg/mL | 3.0 mg/mL |
| pH = 4.80 | Deposits in the vial | Deposits in the vial | Deposits in the vial | Deposits in the vial |
| pH = 4.13 | Partially solubilized | Partially solubilized | Deposits in the vial | Deposits in the vial |
| pH = 3.76 | Almost fully solubilized | Partially solubilized | Deposits in the vial | Deposits in the vial |
| pH = 3.30 | Solubilized | Solubilized | Solubilized | Solubilized |

As noted in Table 2, the solubility of TTX in aqueous AA solutions was found to increase as the pH is lowered. A 0.02M acetic acid solution at pH 3.30 was found to readily dissolve TTX at up to 3.0 mg/ml.

This study confirms the necessity of some water content in a solution for dissolving TTX at the target concentrations.

### Example 3—Solubility of TTX in Water and Acetic Acid and Organic Solvent

In these tests, an acetic acid (“AA”) solution, at pH 3.30, was mixed with an organic solvent at volume ratios of 10%AA/90% organic and 5% AA/95% organic. The organic solvents studied were ethanol (“EtOH”), N,N-Dimethylacetamide (“DMA”), Polyethylene Glycol 400 (“PEG 400”), and Propylene Glycol (“PG”). TTX was added at a concentration of 3.0mg/ml. The aim of the tests was to arrive at a TTX formulation having a pH of 4.0±0.5. Table 3 provides the pH measurements of the solutions before and after TTX addition.

TABLE 3

| | pH | pH with TTX | pH | pH with TTX |
|---|---|---|---|---|
| | (5% | (5% | (5% | (5% |
| Solvent | aq:95% org) | aq:95% org) | aq:95% org) | aq:95% org) |
| EtOH | 5.43 | 6.98 | 5.19 | 7.16 |
| DMA | 7.81 | 9.55 | 7.82 | 10.29 |
| PEG 400 | 6.78 | 8.06 | 6.71 | 7.52 |
| PG | 4.21 | 6.10 | 4.20 | 6.36 |

As noted above, the addition of TTX resulted in a significant upward shift of the pH of the formulation (by roughly 2 pH units). To reduce the pH to the desired value of 4, the formulations were titrated with addition of a 0.696M AA solution. The results of one of these titration tests are presented in Table 4.

In these tests, a solution of TTX in AA (0.02M), at a TTX concentration of 3.0 mg/ml, was added to organic solvents in a v/v amount of 10% aq:90% org to provide a total TTX concentration of 300 μg/ml in the final formulation. The formulations were then titrated to pH 4 by addition of 0.696M AA solution.

TABLE 4

| | | pH after | AA Added | [AA] after | |
|---|---|---|---|---|---|
| Solvent | Initial pH | titration | (mL) | titration (M) | % w/v AA |
| EtOH | 6.79 | 3.92 | 1.1 | 1.726 | 1.0 |
| DMA | 9.69 | 4.00 | 2.4 | 3.369 | 2.0 |
| PEG 400 | 8.58 | 3.99 | 1.2 | 1.866 | 1.1 |
| PG | 6.25 | 4.00 | 0.1 | 0.174 | 0.1 |

As discussed above, the addition of TTX was found to raise pH values beyond the target of 4. Consequently, additional AA was required, with the required AA amount varying based on the organic solvent that was used.

### Example 4—Solubility of TTX in Water and MSA and Organic Solvent

A solubility study similar to Example 3 was conducted by substituting methanesulfonic acid (“MSA”) for AA. As is known, the pKa of MSA is −1.86, whereas the pKa of AA is 4.76. In this study, TTX was dissolved in a solution of 10% MSA solution (0.01 M, pH 2) to arrive at a TTX concentration of 3 mg/ml. This TTX solution was then combined with an organic solvent at 1:9 (v/v) ratio, to result in a formation having 10% aq:90% org (as in Example 3). This results in a formulation having a TTX concentration of 300 μg/ml. As with Example 3, the initial pH of each mixture was measured and then titrated with MSA to reach a pH of 4±0.5. The results of this study are presented in Table 5.

TABLE 5

| | | pH after | | |
|---|---|---|---|---|
| Solvent (*) | Initial pH | titration | MSA added | % w/v MSA |
| DMA | 5.23 | 3.14 | 1 drop MSA conc. | ≈0.01 |
| PEG 400 | 5.33 | 3.69 | 0.5 mL MSA 0.01M | ≈0.01 |
| PG | 3.54 | N/A | N/A | ≈0.01 |

(* In this study, EtOH was not used an organic solvent as TTX was found to
precipitate in the EtOH mixture when stored at 4° C.)

As noted in Table 5, the pH of each of the TTX formulations was found to be lower than formulations prepared with AA. As a result, only a negligible amount of additional MSA was needed to lower the pH of the formulation to the target value. With PG as the organic solvent, the starting pH of the formulation was found to be within the target range of 4±0.5 and, as such, no MSA titration was necessary.

This study shows that with MSA, being a strong acid (pka=−1.86), pH control of TTX formations in organic solvents is more easily controlled.

### Summary of Solubility Studies

The studies that were conducted confirm that TTX is insoluble in mixtures of organic solvents and acids. In other the studies conducted by the inventor, it was found that the solubility of TTX in pure organic solvents (such as EtOH, DMA, PEG, PG) is below 30 μg/ml, which is what was expected. The above findings indicate that the solubility of TTX in a combination of organic solvents and acids is also below 30 μg/ml.

These studies also confirm that water is a needed component in the formulation for obtaining the above-mentioned concentration values of TTX. For example, TTX was found to readily dissolve up to 3 mg/ml in aqueous acetic acid solutions.

In an experiment involving a formulation comprised of 10% aqueous+90% organic solvents (i.e., EtOH, DMA, PEG 400 and PG), and titrating with AA, TTX was found to be solubilized at 0.3 mg/mL. However, the addition of TTX was found to raise the pH of the formulation by approximately 2 pH units. To reach the target pH of about 4.0, addition of 1.0%, 2.0%, 1.1%, and 0.1% AA (all w/v) was found to be required for formulations having 10% Aqueous+90% of EtOH, DMA, PEG 400, and PG, respectively.

In another experiment using 10% aqueous+organic solvents (EtOH, DMA, PEG 400 and PG) and titrating instead with MSA, TTX was found to be solubilized at 0.3mg/mL. The amount of MSA required to achieve the target pH of about 4.0 was less than that for AA. This is believed to be attributed to MSA being a stronger acid than AA.

### B) Stability Studies

### Example 5—Stability of TTX in Formulations of Aqueous Acid (MSA/AA) and Organic Solvent (10% Aqueous:90% Organic (v/v))

In this study, seven formulations were prepared comprising an organic solvent (ethanol (“EtOH”), N, N-Dimethylacetamide (“DMA”), Polyethylene Glycol 400 (“PEG 400”), or Propylene Glycol (“PG”)), an acid (MSA or AA), and water. These formulations were prepared at volume ratios of 10% aqueous component:90% organic component, with a target pH of 4, and a TTX concentration of 300 μg/ml. The samples were prepared and stored in closed vials in a stability testing chamber (40° C. and 75% relative humidity, “RH”) for 24 hours, 48 hours, and 7 days, respectively. Measurement of TTX concentration in the samples was performed using HPLC and calculating the areas under the respective curves. The TTX measurements are reported as a percentage based on the quantified amount of TTX at T0 (i.e., prior to the storage periods). More specifically, the stability of TTX in the sample solutions was calculated as a % Recovery by comparing the peak area of TTX at the respective time points to the peak area of TTX obtained at T=0, according to the following formula:

$$\% Recovery = ( {r}_{U} / {r}_{S} ) \times 100$$

Where: rᵤ=peak response of Tetrodotoxin from the Sample solution at different time points; and rₛ=peak response of Tetrodotoxin from the Sample solution at T=0.

The results of this study are presented in Table 6, where TTX % Recovery data is shown.

TABLE 6

| Sample | T0 | T1 (24 h) | T2 (48 h) | T3 (7 days) |
|---|---|---|---|---|
| EtOH/AA | 100.0% | 97.4% | 98.1% | 92.4% |
| DMA/AA | 100.0% | 97.3% | 96.3% | 92.6% |
| PEG400/AA | 100.0% | 91.3% | 92.8% | 87.3% |
| PG/AA | 100.0% | 92.6% | 92.8% | 90.5% |
| DMA/MSA | 100.0% | 96.3% | 99.4% | 97.9% |
| PEG400/MSA | 100.0% | 90.2% | 91.5% | 97.4% |
| PG/MSA | 100.0% | 89.0% | 90.9% | 90.7% |

The concentrations of TTX indicated in Table 6 are presented as normalized values with respect to the concentration at T0.

In this study, it was found that TTX was sufficiently soluble up to at least a concentration of 300 μg/ml in each of the formulations at pH 4. As can be seen, formulations comprising MSA were found to generally provide better stability results than formulations with AA. This may be attributed to MSA being a stronger acid. While some variation in the stability results was found, there was no clear differentiation amongst the four organic solvents that were reviewed. This suggests that degradation of TTX observed in this study was the result of the 10% aqueous component.

The study also revealed that formulations comprising MSA with PEG 400 or DMA were found to provide the highest stability results.

### Example 6—Stability of TTX in Formulations of Aqueous Acid (MSA/AA) and Organic Solvent (5% Aqueous:95% Organic (v/v))

Based on the findings in Example 5, studies were conducted to investigate TTX solubility and stability with less than 10% water content. In this study, similar tests as above were conducted with formulations comprising a 5% aqueous component and a 95% organic component. In these studies, PEG 400, DMA, and PG were used as the organic solvents and MSA and AA were used as the acids.

Initially, six samples of formulations were attempted with each containing 0.3 mg/ml (or 300 μg/ml) TTX in the acid/organic composition. However, for these solutions, the TTX was found to not fully dissolve when mixed with either AA or 0.01M MSA. Only the sample comprising TTX mixed with 3.48M AA was found to achieve the desired TTX concentration. It was therefore decided to reduce the TTX concentration in the final formulation to 0.15 mg/ml.

To achieve the 5% aq/95% org ratio, 3 mg of TTX was dissolved in 1 ml of either AA or 0.01M MSA and this aqueous solution was mixed with 19 ml of the organic component, comprising DMA, PEG 400, or PG. For the AA samples where TTX did not fully dissolve, 3.48M AA was added until dissolution was achieved. The final concentration of TTX in the samples was 0.15 mg/ml.

In addition, three further samples were prepared containing PEG 400/MSA with one a stabilizer chosen from guanidine, lactose monohydrate, and sodium metabisulfite. These stabilizers were provided up to a concentration of 1 mM.

The above-mentioned samples were kept overnight under refrigeration without any precipitate being found. The sample were then moved to a stability chamber (40° C. and 75% RH) and kept for 24 hours (T1), 120 hours (T2), 168 hours (T3), or 312 hours (T4). The samples were then analyzed by HPLC to measure the amount of TTX. As above, the measurements were performed by calculating peak areas and comparing same with the measurements at T0. The results (expressed as % Recovery) of the TTX measurements are provided in Table 7.

TABLE 7

| | Time (hours) | | | | |
|---|---|---|---|---|---|
| | 0 | 24 | 120 | 168 | 312 |
| Sample | (T0) | (T1) | (T2) | (T3) | (T4) |
| DMA/AA | 100.0% | 99.0% | 93.9% | 92.1% | 86.7% |
| PEG 400/AA | 100.0% | 104.7% | 101.2% | 98.7% | 96.2% |
| PG/AA | 100.0% | 107.3% | 99.8% | 100.1% | 94.4% |
| DMA/MSA | 100.0% | 101.0% | 98.2% | 97.9% | 96.0% |
| PEG 400/MSA | 100.0% | 71.2% | 30.7% | 25.7% | 20.7% |
| PG/MSA | 100.0% | 99.9% | 95.3% | 93.7% | 89.1% |
| PEG 400/MSA/guanidine | 100.0% | 77.7% | 39.3% | 32.8% | 27.1% |
| PEG 400/MSA/lactose | 100.0% | 88.9% | 82.1% | 80.9% | 81.2% |
| PEG 400/MSA/ | 100.0% | 99.9% | 97.0% | 96.1% | 93.3% |
| metabisulfite | | | | | |
| PEG 400/MSA (repeat) | 100.0% | 175.1% | 185.6% | 193.2% | |
| PEG 400/MSA/guanidine | 100.0% | 99.9% | 98.2% | 97.1% | |
| (repeat) | | | | | |

As noted in Table 7, the DMA/MSA sample was found to be relatively stable, whereas the results for PEG 400/MSA and PEG 400/MSA/guanidine were found to be extremely low. The latter was unexpected given that this was not reflected in the results from the 10% aq/90% org tests. Given that both these samples exhibited the same degradation trend, it was postulated that these unexpected results may be attributed insufficient mixing of the sample and to issues with over titration during sample preparation. As above, acid was added to the final formulation to bring the final pH to 4. This was confirmed by subsequent pH measurement of the test samples, where it was determined that the pH of the PEG 400/MSA and PEG 400/MSA/guanidine samples were 0.92 and 0.81, respectively. Thus, the acid titration step did not have an accurate pH measurement. For this reason, the tests comprising PEG 400/MSA and PEG400/MSA/guanidine were repeated and these results are provided at the bottom of Table 7. The results for PEG 400/MSA were found to again include some error in TTX measurement and it is believed that this associated with the high viscosity of the formulation given the 5% aqueous content. As a result, difficulties were encountered with achieving sufficient mixing, in the pH titration step. In addition, the HPLC peaks with this sample were difficult to measure. Thus, the results suggest that using PEG 400 alone with a high organic solvent content (i.e., 95% ) in the subject formulations, or at least the formulations comprising MSA, would be difficult to formulate. These results indicate that it would be preferable to include PG in the formulation to serve as a diluent for the PEG 400.

As shown at the bottom of Table 7, the repeat of the PEG 400/MSA/guanidine test was found to provide desired results.

From this study, it was determined that obtaining a TTX concentration of 300 μg/ml was not feasible for a formulation comprising 5% aqueous and 95% organic components. As such, a target of 150 μg/ml is preferable for this type of formulation.

### Example 7—Stability of TTX in Formulations of Aqueous Acid (MSA/AA) and Organic Solvent Combinations (5% Aqueous:95% Organic (v/v))

In this study, a 5% aqueous/95% organic volume ratio was again used for the sample formulations. However, in this instance the organic phase was formed with a combination of solvents. This test was conducted in view of the concentration limitation of 95% for solvents. In other words, no one solvent can exceed a concentration of 95% in the formulation.

For this purpose, the following formulations were designed:
F1: aqueous/DMA/PG=5%/25%/70%
F2: aqueous/DMA/PEG 400=5%/25%/70%
F3: aqueous/PG/PEG 400=5%/50%/45%

For the aqueous acid component, AA and MSA were investigated. However, formulations comprising F1+AA and F2+AA were not considered in view of the large amount of AA that was needed to lower the pH to the desired level. The formulations studied are listed in Table 8.

TABLE 8

| Sample | Composition | Acid | Organic | % aqueous | % organic |
|---|---|---|---|---|---|
| 1 | F3 + AA | AA | PG/PEG 400 | 5 | 50/45 |
| 2 | F1 + MSA | MSA | DMA/PG | 5 | 25/70 |
| 3 | F2 + MSA | MSA | DMA/PEG 400 | 5 | 25/70 |
| 4 | F3 + MSA | MSA | PG/PEG 400 | 5 | 50/45 |
| 5 | F3 + MSA + | MSA | PG/PEG 400 | 5 | 50/45 |
| | sodium | | | | |
| | metabisulfite | | | | |

Controls used in these tests comprised samples that only comprised an aqueous phase.

Preliminary stability data for these samples was obtained after maintaining the samples at 25° C. and 40% RH for one week (T1) and this data is summarized in Table 9.

TABLE 9

| | | | T1 |
|---|---|---|---|
| Sample | Composition | T0 | (7 days) |
| 1 | F3 + AA | 100.0% | 100.2% |
| 2 | F1 + MSA | 100.0% | 99.7% |
| 3 | F2 + MSA | 100.0% | 100.1% |
| 4 | F3 + MSA | 100.0% | 100.0% |
| 5 | F3 + MSA + Metabisulfite | 100.0% | 99.0% |

As noted, all the samples were found to maintain TTX stability for up to 7 days under the above-mentioned conditions.

For the next section of this study, only samples 1, 3, and 4 were utilized and these samples were subjected to stability testing under the following three different conditions and time periods:
1) [25° C., 40% RH, 7 days]+[40° C., 75% RH, 7 days];
2) [25° C., 40% RH, 7 days]+[40° C., 75% RH, 16 days];
3) [25° C., 40% RH, 7 days]+[40° C., 75% RH, 24 days];
4) 25° C., 40% RH, 8 weeks; and
5) 2-8° C., ambient RH, 6 months.

The results of these tests are provided in Tables 10, 11, and 12.

TABLE 10
Stability test conditions 1 to 3
25° C./40% RH for 1 week, then transfer to 40° C./75%RH

| | | | | 1 wk 25° C. + | | 1 wk 25° C. + | | 1 wk 25° C. + | |
|---|---|---|---|---|---|---|---|---|---|
| Sample | T0 | 1 wk 25° C. | | 7 days 40° C. | | 16 days 40° C. | | 24 days 40° C. | |
| composition | pH | % TTX | pH | % TTX | pH | % TTX | pH | % TTX | pH |
| F3 + AA | 4.47 | 100.2% | 4.49 | 96.4% | NA | 91.2% | 4.17 | 87.3% | 4.59 |
| F2 + MSA | 3.89 | 100.1% | 4.01 | 97.0% | NA | 91.5% | 5.64 | 86.3% | 5.68 |
| F3 + MSA | 4.32 | 100.0% | 4.72 | 97.3% | NA | 92.1% | 5.29 | 89.2% | 5.23 |

TABLE 11
Stability test condition 4
25° C./40% RH

| Sample | T0 | 1 week | | 4 weeks | | 8 weeks | |
|---|---|---|---|---|---|---|---|
| composition | pH | % TTX | pH | % TTX | pH | % TTX | pH |
| F3 + AA | 4.47 | 100.2% | 4.49 | 99.0% | 4.35 | 96.9% | 4.60 |
| F2 + MSA | 3.89 | 100.1% | 4.01 | 97.6% | 4.97 | 90.9% | 4.86 |
| F3 + MSA | 4.32 | 100.0% | 4.72 | 98.5% | 5.28 | 97.1% | 5.20 |

TABLE 12
Stability test condition 5
2-8° C./Ambient RH

| | T0 | 1 month | | 2 months | | 3 months | | 6 months | |
|---|---|---|---|---|---|---|---|---|---|
| Sample composition | pH | % TTX | pH | % TTX | pH | % TTX | pH | % TTX | pH |
| F3 + AA | 4.47 | 100.2% | 4.45 | 99.8% | 4.51 | 94.3% | 4.53 | 98.9% | 4.75 |
| F2 + MSA | 3.89 | 99.2% | 3.95 | 91.5% | 3.98 | 95.7% | 3.98 | 91.1% | 4.21 |
| F3 + MSA | 4.32 | 97.0% | 4.93 | 99.4% | 4.76 | 99.3% | 4.82 | 98.8% | 4.99 |

In the above results, the % TTX was measured using HPLC and quantified by peak area as with previous examples. The TTX amounts are expressed with reference to the amount at T0.

In the above studies, it was found that the amount of TTX in the controls (lacking an organic component) fell below 90% within 5-7 days under conditions of 40° C. and 75% RH. In contrast, the samples tested with 5% aqueous and 95% organic component were found to have a TTX amount that was 90% of T0 up to approximately 20 days under such accelerated conditions.

### Example 8—Stability of TTX in Formulations of Aqueous Acid and Organic Solvent Combination (2% Aqueous:98% Organic (v/v))

Similar to Example 7, a study was conducted to determine the solubility and stability characteristics of TTX in formulations where the aqueous component was reduced to 2% (v/v), with the remaining 98% (v/v) of the formulation comprising the organic component. The organic component being comprised of a combination of organic solvents as in the previous example.

In this study, formulations were prepared with three buffer systems, namely, acetate, citrate, and phosphate buffer systems. Formulations were prepared having 2% aqueous solution and 98% organic solvent, comprising 50% (v/v) PG and 48% (v/v) PEG. Aqueous MSA was used to titrate the formulations to pH 4, 5, and 6. TTX samples were then prepared at a concentration of 60 μg/ml. The formulations were adapted with the buffer to remain at about pH 4 to minimize the amount of MSA required after TTX addition.

The samples were prepared by dissolving TTX in pH 4 buffer solutions and the solvent was added. Upon complete dissolution of TTX, the pH was adjusted with aqueous MSA to a pH of 4 or 6. Table 13 summarizes the samples that were prepared in this study.

TABLE 13

| | | Buffer | | |
|---|---|---|---|---|
| Samples | TTX (mg) | pH 4 (mL) | PG (mL) | PEG (mL) |
| TTX-Acetate pH 4 | 3 | 1 | 25 | 24 |
| TTX-Acetate pH 6 | 3 | 1 | 25 | 24 |
| TTX-Citrate pH 4 | 3 | 1 | 25 | 24 |
| TTX-Citrate pH 6 | 3 | 1 | 25 | 24 |
| TTX-Phosphate pH 4 | 3 | 1 | 25 | 24 |
| TTX-Phosphate pH 6 | 3 | 1 | 25 | 24 |

Corresponding control samples were also prepared. The formulations were then transferred to three different stability chambers to assess the stability of the formulations under the following three conditions: 40° C./75% RH; 25° C./40% RH; and 2-8° C. The samples were then assayed at varying times depending on the conditions. Table 14 summarizes the assay times for the samples.

TABLE 14

| Conditions | Assay points | | | | | | Time Units |
|---|---|---|---|---|---|---|---|
| 40° C./75% RH | T0 | T3 | T7 | T14 | T21 | T28 | Days |
| 25° C./40% RH | T0 | T2 | T4 | — | — | — | Weeks |
| 2-8° C. | T0 | T1 | T6 | — | — | — | Months |

Tables 15, 16, and 17 summarize the results of these studies.

TABLE 15
Stability test: 40° C./75% RH

| Time | % Recovery vs T0 | | | | | | pH measurements | | | | | |
|---|---|---|---|---|---|---|---|---|---|---|---|---|
| (days) | T0 | T3 | T7 | T14 | T21 | T28 | T0 | T3 | T7 | T14 | T21 | T28 |
| TTX- | 100.0% | 88.5% | 87.2% | 74.2% | 62.6% | 58.1% | 4.04 | 1.94 | 1.79 | 1.77 | 1.88 | 1.92 |
| Acetate pH | | | | | | | | | | | | |
| 4 | | | | | | | | | | | | |
| TTX- | 100.0% | 93.3% | 95.6% | 93.1% | 83.6% | 75.0% | 6.02 | 5.91 | 5.86 | 5.77 | 5.72 | 5.66 |
| Acetate pH | | | | | | | | | | | | |
| 6 | | | | | | | | | | | | |
| TTX-Citrate | 100.0% | 107.1% | 107.8% | 108.5% | 100.3% | 95.0% | 4.11 | 4.26 | 4.14 | 4.2 | 4.29 | 4.13 |
| pH 4 | | | | | | | | | | | | |
| TTX-Citrate | 100.0% | 101.1% | 102.3% | 100.6% | 89.2% | 84.0% | 5.79 | 5.9 | 5.8 | 5.84 | 5.89 | 5.82 |
| pH 6 | | | | | | | | | | | | |
| TTX- | 100.0% | 103.5% | 99.7% | 99.5% | 89.5% | 83.2% | 5.61 | 4.55 | 4.53 | 4.48 | 4.46 | 4.46 |
| Phosphate | | | | | | | | | | | | |
| pH 4 | | | | | | | | | | | | |
| TTX- | 100.0% | 102.8% | 98.8% | 96.9% | 85.5% | 76.8% | 4.48 | 5.6 | 5.56 | 5.52 | 5.51 | 5.45 |
| Phosphate | | | | | | | | | | | | |
| pH 6 | | | | | | | | | | | | |

TABLE 16
Stability test: 25° C./40% RH

| | % Recovery vs T0 | | | pH measurements | | |
|---|---|---|---|---|---|---|
| Time (weeks) | T0 | T2 | T4 | T0 | T2 | T4 |
| TTX-Acetate pH 4 | 100.0% | 100.5% | 93.1% | 4.04 | 1.81 | 1.74 |
| TTX-Acetate pH 6 | 100.0% | 103.8% | 96.4% | 6.02 | 5.82 | 5.83 |
| TTX-Citrate pH 4 | 100.0% | 114.1% | 106.0% | 4.11 | 4.14 | 4.16 |
| TTX-Citrate pH 6 | 100.0% | 109.2% | 102.0% | 5.79 | 5.76 | 5.77 |
| TTX-Phosphate pH 4 | 100.0% | 108.6% | 100.9% | 5.61 | 4.48 | 4.46 |
| TTX-Phosphate pH 6 | 100.0% | 108.3% | 100.4% | 4.48 | 5.60 | 5.55 |

TABLE 17
Stability test: 2-8° C./40% RH

| | % Recovery vs T0 | | | pH measurements | | |
|---|---|---|---|---|---|---|
| Time (weeks) | T0 | T1 | T6 | T0 | T1 | T6 |
| TTX-Acetate pH 4 | 100.0% | 101.1% | 88.0% | 4.04 | 1.85 | 1.91 |
| TTX-Acetate pH 6 | 100.0% | 97.8% | 99.3% | 6.02 | 5.91 | 5.91 |
| TTX-Citrate pH 4 | 100.0% | 107.2% | 101.0% | 4.11 | 4.1 | 4.13 |
| TTX-Citrate pH 6 | 100.0% | 103.9% | 96.6% | 5.79 | 5.72 | 5.66 |
| TTX-Phosphate pH 4 | 100.0% | 104.0% | 100.8% | 5.61 | 4.47 | 4.45 |
| TTX-Phosphate pH 6 | 100.0% | 103.6% | 99.8% | 4.48 | 5.57 | 5.56 |

As noted in Tables 15-17, the TTX-Acetate, pH 4 samples were found to exhibit a reduction in pH over time. This is believed to be the result of acetic acid being a relatively weak acid and the acetic acid/acetate buffer being relatively slower in accommodating pH variations. Consequently, pH a titration was rendered difficult to perform. It is also possible that insufficient mixing of the samples may have also contributed to the unexpected pH measurements. In contrast, citric acid and phosphoric acids are stronger acids, therefore, pH equilibrium was achieved more quickly. Thus, these studies suggest that using a stronger acid (such as MSA) is desirable to maintain more efficient pH control.

In this study, some of the chromatographic integration and quantification were found to be unreliable due to imperfect TTX peak shapes. This is believed to be the result of the high organic solvent concentration in the formulations. Thus, although TTX formulations having an aqueous component as low as 2% are found to be stable (as noted below), measurement of the TTX concentration may be challenging with such high organic concentration.

This study shows that, for formulations having a 2% aqueous component, TTX solubility is at least about 60 μg/ml. Further, the data from the 2% aqueous samples placed in accelerated conditions (i.e., 40° C.) did not show a significant difference from the 5% aqueous samples placed under the same conditions. This therefore suggests that having an aqueous component as low as 2% is still sufficient to maintain TTX stability. This conclusion is further supported by the stability data obtained from the tests run at 2-8° C. These data therefore lead to the conclusion that at least 2% or 5% aqueous composition in the formulations is sufficient to maintain the stability of TTX.

### Summary of Stability Studies

Formulations were studied comprising 10% (v/v) aqueous and 90% (v/v) organic components. These formulations comprised an aqueous phase of an acid (AA or MSA) solution. The organic phase comprised EtOH, DMA, PEG400, or PG. The formulations were prepared with pH of about 4 and TTX concentrations of 300 μg/ml were achieved. The stability of TTX in these formulations persisted for approximately 7 days with some variability. Owing to the high organic solvent content, chromatographic effects such as peak splitting made it challenging to obtain results using the HPLC method that was employed.

These studies also revealed that MSA is a better acid to use than AA in view of MSA being a stronger acid (as indicated by its pKa). The studies also revealed that there was no clear differentiation between the organic solvents used in terms of TTX stability.

Based on the favorable results from the 10% (v/v) aqueous studies, further studies were conducted using a lower (5% and 2% ) aqueous component. As indicated above, studies were conducted with a formulation having a 5% aqueous and 95% organic composition, wherein AA and MSA were employed as the acid, PEG 400, DMA, and PG employed as organic solvents, guanidine HCl, lactose monohydrate, and sodium metabisulfite as stabilizers. With these formulations, TTX was found to be soluble up to about 150 ug/mL and TTX stability was found to be improved as compared to the 10% aqueous studies.

In view of the encouraging data from the 5% (v/v) aqueous studies, further experiments were conducted using the 5% aqueous and 95% organic composition, with AA and MSA used as acids, DMA, PEG, PG, and combinations of two solvents, used as the organic component. Guanidine HCl, SBECD, and sodium metabisulfite were used as stabilizers. Three stability conditions were investigated: 40° C./75% RH (1 month), 25° C./40% RH (2 months), and 2-8° C. (6 months). TTX values were found to be stable (i.e., remaining above 90% ) for up to approximately 20 days. The stability data at 2-8° C. indicate that TTX may not follow the conventional Arrhenius estimate of temperature vs degradation reaction rate. This data, therefore, suggests that TTX formulations can remain stable at low aqueous contents.

Based on the favorable 5% aqueous data, 2% (v/v) aqueous formulations were explored to determine if improved stability could be achieved. In these studies, TTX solubility was found to be about 60 μg/mL. Stability testing conducted at 40° C. did not show a significant difference between 2% and 5% aqueous compositions. This suggests that stable TTX formulations can be achieved with aqueous concentrations as low as 2%. However, chromatographic defects encountered during the analytical phase were challenging for the 2% aqueous formulation.

### Example 9—Stability of TTX in Formulations with Various PG/PEG 400 Ratios and pH

In this study, experiments were conducted to examine the effects of different PG and PEG 400 ratios (organic solvents) on TTX stability. Experiments were also conducted to examine the effect of formulation pH on TTX stability. In these studies, aqueous MSA was used for the acid component.

The formulations were made up with a TTX concentration of 100 μg/ml and according to the volume ratios of MSA/PG/PEG 400 listed in Table 18. Where MSA is 0.01M aqueous MSA.

TABLE 18

| Expt. | | | | | |
|---|---|---|---|---|---|
| No. | TTX | MSA | PG | PEG 400 | pH |
| 1 | 100 μg/ml | 5 | 40 | 55 | 5 |
| 2 | 100 μg/ml | 5 | 50 | 45 | 5 |
| 3 | 100 μg/ml | 5 | 60 | 35 | 5 |
| 4 | 100 μg/ml | 5 | 70 | 25 | 5 |
| 5 | 100 μg/ml | 5 | 80 | 15 | 5 |
| 6 | 100 μg/ml | 5 | 50 | 45 | 6 |
| 7 | 100 μg/ml | 5 | 50 | 45 | 4 |
| 8 | 100 μg/ml | 5 | 50 | 45 | 3 |
| 9 | 100 μg/ml | 5 | 80 | 15 | 6 |
| 10 | 100 μg/ml | 5 | 80 | 15 | 4 |
| 11 | 100 μg/ml | 5 | 80 | 15 | 3 |
| 12 | 100 μg/ml | 5 | 30 | 65 | 5 |
| 13 | 100 μg/ml | 5 | 20 | 75 | 5 |

Table 18 also lists the initial pH of the formulations. In the course of preparing the samples, it was noted that increasing PG amounts resulting in lowering of pH.

The samples were subjected to stability tests under 40° C. and 75% RH, with samples assayed at t=0, 2 weeks, and 4 weeks.

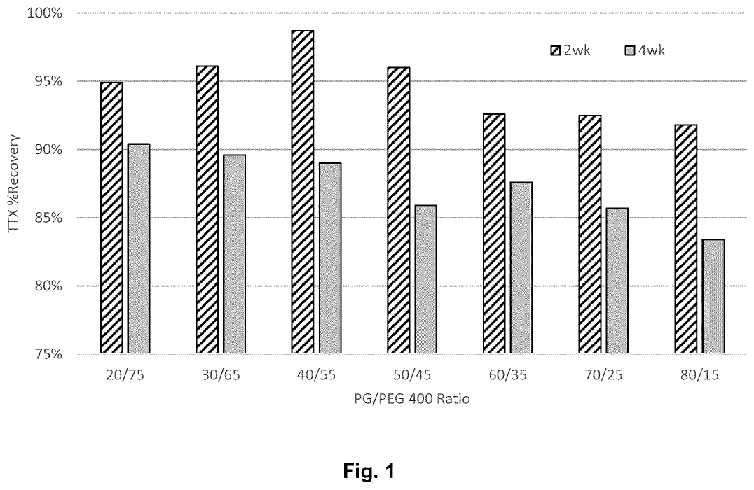

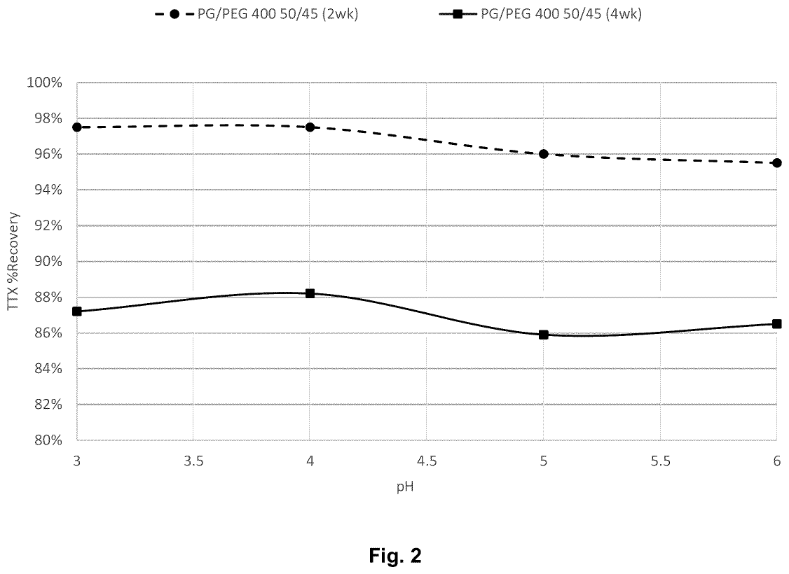

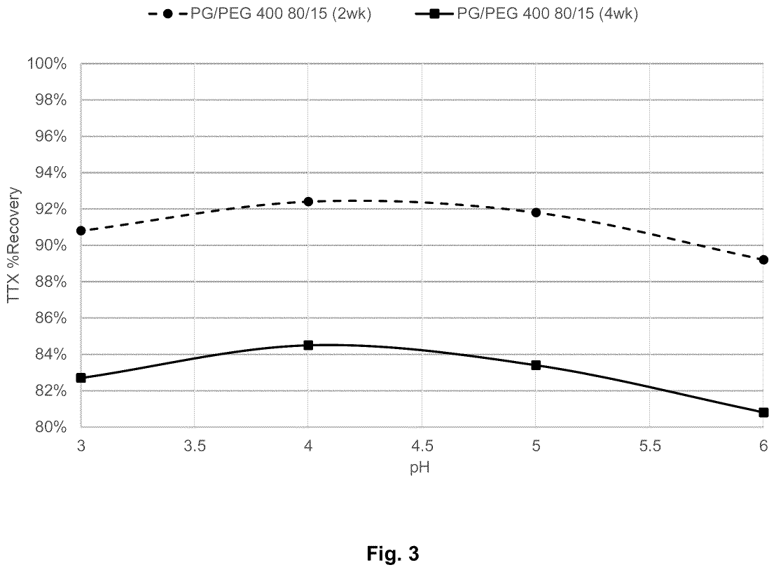

The results from these tests are presented in FIGS. 1, 2 and 3. It was surprisingly noted that a higher PEG 400 amount resulted in improved TTX stability for both stability testing environments. Without being bound to any theory, it is believed that this finding of improved TTX stability is the result of PEG 400 (as compared to PG) being better able to bind water molecules and thereby reduce the exposure of TTX to the water component in the formulation.

In this regard, it was previously shown that, in a PEG aqueous solution, one water molecule forms hydrogen bonds with one monomer of the PEG chain when water is present in a high concentration. However, in the case of a low water concentration, one water molecule forms hydrogen bonds with two monomers of the PEG chain. See P. Molyneux (Synthetic polymers. In Water—A Comprehensive Treatise; Franks, F., Ed.; Plenum Press: New York, 1975; Vol. 4.) and S. Lusse et al (Macromolecules 1996, 29, 4251-4257). 2

It was also noted that a formulation having a pH of about 3 to about 6 also exhibited improved TTX stability. Of this range, a pH of about 4 was found to be ideal for TTX stability.

In this study, as with the other studies discussed above, stability was also measured at conditions of 2-8° C. As with the earlier data, the TTX formulations stored under these conditions were found to be stable. These data indicate that TTX formulation stability is influenced by storage temperature.

In separate studies that were conducted, it was also noted that PEG 400 increases formulation viscosity and that the formulation viscosity at 5° C. is roughly 3-4 times greater that at 25° C. Thus, while the use of PEG in TTX formulations has been found to be preferable, the manner of administration of the formulation may need to be adjusted where the PEG concentration is higher, such as by use of larger gauge needles.

Taking the above factors into consideration, it was determined that formulation 2 would be ideal for further investigation. This formulation comprised MSA/PG/PEG 400 in the ratio 5/50/45 (v/v) (where MSA is 0.01M aqueous MSA).

The study described in Example 9 was repeated to provide supplementary data regarding impurities. The relative amounts of various degradation products of TTX were measured at the noted time points. The degradation products of TTX are identified as impurities g-TTX, o-TTX, e-TTX and a-TTX as shown in Table 19.

TABLE 19

| | | Molecular | Impurity |
|---|---|---|---|
| Chemical Name | Structure and Formula | Weight | Origin |
| 4,9-anhydro- tetrodotoxin (a-TTX) | 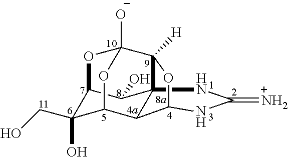 | 301 | Degradation product |
| | C11H15N3O7 | | |
| 4-epi-tetrodotoxin (e-TTX) | 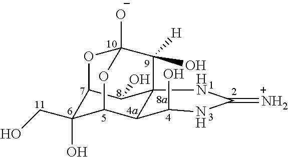 | 319 | Degradation product |
| | C11H17N3O8 | | |
| 4-olefinic- tetrodotoxin (o-TTX) | 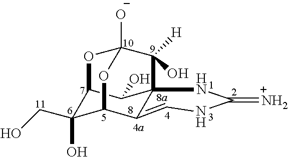 | 301 | Degradation product |
| | C11H15N3O7 | | |
| 11- galactopyranosyl- tetrodotoxin (g-TTX) | 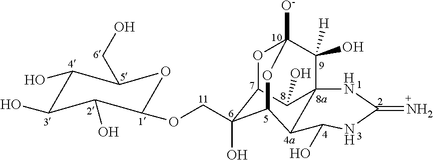 | 481 | drug substance impurity from the manufacturing process, not a degradation product |
| | C17H27N3O13 | | |

The study results are shown in Table 20 below:

TABLE 20

| | | | Impurities T0 | | | | | |
|---|---|---|---|---|---|---|---|---|
| Sample | | | Other | | | | | |
| ID | PG:PEG | pH | Impurities | g-TTX | o-TTX | e-TTX | a-TTX | Sum |
| 29 | 20:75 | 5 | — | 0.168 | — | — | — | 0.168 |
| 28 | 30:65 | 5 | — | 0.269 | — | — | 0.135 | 0.404 |
| 1 | 40:55 | 5 | — | 0.343 | — | — | 0.134 | 0.477 |
| 2 | 50:45 | 5 | — | 0.399 | — | — | 0.203 | 0.602 |
| 3 | 60:35 | 5 | — | 0.445 | — | 0.055 | 0.247 | 0.747 |
| 4 | 70:25 | 5 | 0.162 | 0.458 | 0.054 | 0.085 | 0.246 | 1.005 |
| 5 | 80:15 | 5 | 0.260 | 0.465 | — | — | 0.250 | 0.974 |
| 6 | 50:45 | 6 | — | 0.411 | — | — | 0.245 | 0.656 |
| 7 | 50:45 | 4 | — | 0.412 | — | 0.068 | 0.200 | 0.680 |
| 8 | 50:45 | 3 | — | 0.406 | — | 0.072 | 0.224 | 0.703 |
| 9 | 80:15 | 6 | 0.248 | 0.456 | — | — | 0.346 | 1.050 |
| 10 | 80:15 | 4 | 0.225 | 0.450 | 0.054 | 0.083 | 0.248 | 1.060 |
| 11 | 80:15 | — | 0.234 | 0.452 | — | 0.075 | 0.289 | 1.049 |
| | | | Impurities, 2W | | | | | |
| Sample | | | Other | | | | | |
| ID | PG:PEG | pH | Impurities | g-TTX | o-TTX | e-TTX | a-TTX | Sum |
| 29 | 20:75 | 5 | — | 0.419 | 0.300 | 0.735 | 4.609 | 6.063 |
| 28 | 30:65 | 5 | — | 0.457 | — | 0.322 | 4.414 | 5.193 |
| 1 | 40:55 | 5 | — | 0.344 | 0.134 | 0.305 | 4.329 | 5.112 |
| 2 | 50:45 | 5 | — | 0.382 | 0.091 | 0.177 | 4.664 | 5.314 |
| 3 | 60:35 | 5 | 0.187 | 0.348 | 0.060 | 0.286 | 5.331 | 6.024 |
| 4 | 70:25 | 5 | 0.249 | 0.361 | 0.044 | 0.275 | 5.772 | 6.452 |
| 5 | 80:15 | 5 | — | 0.507 | — | 0.293 | 7.128 | 7.928 |
| 6 | 50:45 | 6 | — | 0.511 | — | 0.770 | 6.650 | 7.931 |
| 7 | 50:45 | 4 | — | 0.353 | 0.045 | 0.233 | 4.801 | 5.432 |
| 8 | 50:45 | 3 | 0.190 | 0.361 | 0.034 | 0.291 | 5.175 | 5.860 |
| 9 | 80:15 | 6 | — | 0.379 | — | 0.383 | 7.558 | 8.320 |
| 10 | 80:15 | 4 | 0.264 | 0.299 | — | 0.282 | 6.504 | 7.085 |
| 11 | 80:15 | 3 | 0.613 | 0.363 | 0.146 | 0.389 | 7.355 | 8.253 |
| | | | Impurities, 4W | | | | | |
| Sample | | | Other | | | | | |
| ID | PG:PEG | pH | Impurities | g-TTX | o-TTX | e-TTX | a-TTX | Sum |
| 29 | 20:75 | 5 | — | 0.349 | 0.612 | 1.041 | 8.150 | 10.151 |
| 28 | 30:65 | 5 | — | 0.344 | 0.394 | 0.650 | 7.721 | 9.108 |
| 1 | 40:55 | 5 | — | 0.367 | 0.274 | 0.511 | 8.120 | 9.272 |
| 2 | 50:45 | 5 | — | 0.362 | 0.227 | 0.414 | 8.712 | 9.716 |
| 3 | 60:35 | 5 | — | 0.365 | — | 0.533 | 9.749 | 10.647 |
| 4 | 70:25 | 5 | — | 0.353 | — | 0.587 | 10.684 | 11.624 |
| 5 | 80:15 | 5 | 1.841 | 0.562 | — | 0.660 | 11.785 | 14.848 |
| 6 | 50:45 | 6 | — | 0.336 | 0.391 | 0.897 | 10.360 | 11.984 |
| 7 | 50:45 | 4 | 1.027 | 0.432 | 0.126 | 0.543 | 9.624 | 11.753 |
| 8 | 50:45 | 3 | — | 0.400 | — | 0.772 | 10.364 | 11.536 |
| 9 | 80:15 | 6 | 0.448 | 0.378 | 0.178 | 0.657 | 12.829 | 14.490 |
| 10 | 80:15 | 4 | 1.514 | 0.416 | 0.192 | 0.742 | 13.016 | 15.881 |
| 11 | 80:15 | 3 | 2.805 | 0.350 | 0.995 | 0.982 | 13.487 | 18.620 |

The results of this study generally showed consistency compared to the initial study. In particular, the formulation comprising PG:PEG 400: MSA (50:45:5) demonstrates good stability

### Example 10—Stability of TTX in Formulations with Different Aqueous Acids and Different PEG

In this study, the formulation mentioned above, i.e., a formulation comprising MSA/PG/PEG in the ratio 5/50/45 (v/v), was investigated by varying the acids used. For these tests, the formulations were made up with 100 μg/ml TTX. The following acids were investigated: benzenesulfonic acid, formic acid, hydrochloric acid, phosphoric acid, sulfuric acid, sulfurous acid, and citric acid.

As the previous studies have focused on PEG 400, this study further involved an investigation of different PEG components, in particular, PEG 200, PEG 300, and PEG 600. For these experiments, 0.01M aqueous MSA was used as the common acid component and the following comparative formulations were prepared with v/v ratios of MSA/PG/PEG: 5/70/25 (“low” PEG); 5/50/45 (“target” PEG); and 5/30/65 (“high” PEG).

In all cases, the stability tests were conducted under conditions of 40° C. and 75% RH and samples were assayed at t=0, 2 weeks, and 4 weeks.

The combined results of these studies are presented in Table 21. As with the previous data provided above, the % recovery of TTX is presented in Table 21 with respect to the amount of TTX at T0.

TABLE 21

| | T0 | | 2 weeks | | 4 weeks | |
|---|---|---|---|---|---|---|
| | % | | % | | % | |
| Sample | Recovery | pH | Recovery | pH | Recovery | pH |
| Benzenesulfonic | 100.0% | 5.31 | 92.7% | 4.89 | 85.7% | 5.81 |
| acid | | | | | | |
| Formic acid | 100.0% | 4.50 | 93.0% | 4.73 | 85.8% | 4.84 |
| Hydrochloric acid | 100.0% | 4.82 | 92.8% | 5.09 | 84.8% | 4.88 |
| Phosphoric acid | 100.0% | 4.36 | 91.8% | 4.43 | 81.7% | 4.48 |
| Sulfuric acid | 100.0% | 5.14 | 93.3% | 5.00 | 85.1% | 5.18 |
| Sulfurous acid | 100.0% | 4.40 | 92.5% | 4.40 | 84.9% | 4.43 |
| Citric acid | 100.0% | 4.06 | 89.3% | 4.19 | 90.0% | 4.17 |
| PEG 200 (Low) | 100.0% | 4.45 | 88.6% | 4.74 | 78.2% | 4.65 |
| PEG 200 (Target) | 100.0% | 5.37 | 79.6% | 5.60 | 67.2% | 5.64 |
| PEG 200 (High) | N/A* | 5.20 | N/A* | 5.31 | N/A* | 5.28 |
| PEG 300 (Low) | 100.0% | 5.15 | 89.2% | 5.20 | 79.7% | 5.06 |
| PEG 300 (Target) | 100.0% | 5.29 | 89.0% | 5.45 | 80.9% | 5.36 |
| PEG 300 (High) | 100.0% | 5.20 | 93.4% | 4.48 | 85.2% | 4.75 |
| PEG 600 (Low) | 100.0% | 5.28 | 88.8% | 5.04 | 78.7% | 5.04 |
| PEG 600 (Target) | 100.0% | 5.26 | 91.7% | 5.01 | 82.7% | 4.98 |
| PEG 600 (High) | 100.0% | 5.45 | 92.4% | 4.96 | 84.0% | 4.99 |

*The TTX peaks in these samples were unreadable owing to the presence of PEG 200.

As can be seen, after the 4-week examination (under accelerated conditions) none of the tested samples was found to exhibit increased TTX stability over the previously tested formulation comprising MSA:PG:PEG 400 at a v/v ratio of 5:50:45. Most of the tested samples were found to lose approximately 10% of the TTX during the last two weeks of the study; however, the formulation comprising citric acid was an exception.

Benzenesulfonic acid exhibited a high variability in pH measurement and was also generally difficult to use (requiring more care and control as compared to the other acids studied). Thus, it is concluded that this acid may not be an ideal candidate for TTX formulations, primarily owing to the difficulty of pH titration. Phosphoric acid does not appear to improve TTX stabilize as compared to MSA. The formulations containing other acids—formic acid, hydrochloric acid, sulfuric acid, sulfurous acid, and citric acid—showed similar TTX stability to MSA. These studies indicate that the stability of TTX is not related to the acid employed and that various acids may be used for preparing stable TTX liquid formulations.

In comparing TTX stability in formulations comprising PEG 400 with other grades of PEG it is noted that PEG 400 formulations were found to result in greater TTX stability as compared to formulations comprising PEG 200 or PEG 300. While stability using PEG 600 was found to be similar to that achieved with PEG 400, the formulation viscosity was found to be much greater than that using PEG 400. This would be attributed to the higher molecular weight of PEG 600. With these findings, and the fact that PEG 400 is easily accessible, it is believed that PEG 400 would be preferred for the present formulations.

An additional experiment was conducted to further study the effects of the type of acid and PEG on the formulation. The samples as shown in Table 22 below were repeated at target pH 5 for pH/Assay/RS stability at 40° C. at time points T0, 2 weeks and 4 weeks.

In the following tables the term “test” refers to all the chemical tests conducted on each sample at each stability test time point. The term “assay” refers to the test to determine the content of TTX and the term “RS” refers to the “related substances” test which is the test for the presence and amount of impurities.

TABLE 22
Stability study: 40° C./75% RH at T0, 2W, 4W

| Samples | TTX | Acid | PG | PEG | pH | Test |
|---|---|---|---|---|---|---|
| 14 | 100 μg/mL | Benzenesulfonic | 50% | PEG 400: | 5 | assay, |
| | | acid | | 45% | | RS, pH |
| 15 | 100 μg/mL | Formic acid | 50% | PEG 400: | 5 | assay, |
| | | | | 45% | | RS, pH |
| 16 | 100 μg/mL | Hydrochloric | 50% | PEG 400: | 5 | assay, |
| | | acid | | 45% | | RS, pH |
| 17 | 100 μg/mL | Phosphoric acid | 50% | PEG 400: | 5 | assay, |
| | | | | 45% | | RS, pH |
| 18 | 100 μg/mL | Sulfuric acid | 50% | PEG 400: | 5 | assay, |
| | | | | 45% | | RS, pH |
| 19 | 100 μg/mL | Sulfurous acid | 50% | PEG 400: | 5 | assay, |
| | | | | 45% | | RS, pH |
| 20 | 100 μg/mL | Citric acid | 50% | PEG 400: | 5 | assay, |
| | | | | 45% | | RS, pH |
| 21 | 100 μg/mL | MSA | 50% | PEG 200: | 5 | assay, |
| | | | | 45% | | RS, pH |
| 22 | 100 μg/mL | MSA | 50% | PEG 300: | 5 | assay, |
| | | | | 45% | | RS, pH |
| 23 | 100 μg/mL | MSA | 50% | PEG 600: | 5 | assay, |
| | | | | 45% | | RS, pH |

The stability results are for the formulations of Table 22 provided in Tables 23 and 24. Table 23 includes the measured pH values at each time point and the percent of TTX relative to the TTX at T0 and Table 24 includes the relative percentage of the TTX degradation products relative to T0.

TABLE 23

| Sample | Aqueous | | pH measured | | | Assay (mg/mL) | | | Assay (% LC) | | |
|---|---|---|---|---|---|---|---|---|---|---|---|
| ID | Acids | PEGx | T0 | 2W | 4W | T0 | 2W | 4W | T0 | 2W | 4W |
| 14 | Benzenesulfonic | PEG | 5.11 | 4.81 | 4.89 | 0.0980 | 0.0930 | 0.0854 | 100.32% | 95.19% | 87.45% |
| | | 400 | | | | | | | | | |
| 15 | Formic | PEG | 4.56 | 4.70 | 4.75 | 0.0997 | 0.0938 | 0.0842 | 100.31% | 94.36% | 84.73% |
| | | 400 | | | | | | | | | |
| 16 | HCl | PEG | 4.66 | 4.76 | 4.69 | 0.0999 | 0.0948 | 0.0888 | 100.32% | 95.16% | 89.14% |
| | | 400 | | | | | | | | | |
| 17 | Phosphoric | PEG | 4.51 | 4.44 | 4.39 | 0.0990 | 0.0920 | 0.0839 | 100.63% | 93.44% | 85.23% |
| | | 400 | | | | | | | | | |
| 18 | Sulfuric | PEG | 4.94 | 4.88 | 4.68 | 0.0989 | 0.0929 | 0.0858 | 99.35% | 93.31% | 86.15% |
| | | 400 | | | | | | | | | |
| 19 | Sulfurous | PEG | 5.27 | 5.22 | 5.16 | 0.0978 | 0.0913 | 0.0848 | 100.69% | 93.94% | 87.28% |
| | | 400 | | | | | | | | | |
| 20 | Citric | PEG | 4.46 | 4.48 | 4.41 | 0.0957 | 0.0883 | 0.0762 | 97.66% | 90.18% | 77.75% |
| | | 400 | | | | | | | | | |
| 21 | MSA | PEG | 5.53 | 5.05 | 5.04 | 0.1071 | 0.0983 | 0.0896 | 99.52% | 91.35% | 83.27% |
| | | 200 | | | | | | | | | |
| 22 | MSA | PEG | 5.45 | 4.94 | 5.05 | 0.0971 | 0.0912 | 0.0846 | 99.54% | 93.50% | 86.73% |
| | | 300 | | | | | | | | | |

TABLE 24

| | | | Impurities, T0 | | | | | |
|---|---|---|---|---|---|---|---|---|
| Sample | | | Other | | | | | |
| ID | Aqueous Acids | PEGx | impurities | g-TTX | o-TTX | e-TTX | a-TTX | Sum |
| 14 | Benzenesulfonic | PEG | — | 0.401 | — | — | 0.242 | 0.643 |
| | | 400 | | | | | | |
| 15 | Formic | PEG | — | 0.436 | — | 0.111 | 0.318 | 0.865 |
| | | 400 | | | | | | |
| 16 | HCl | PEG | — | 0.427 | — | 0.058 | 0.212 | 0.697 |
| | | 400 | | | | | | |
| 17 | Phosphoric | PEG | — | 0.428 | — | 0.080 | 0.253 | 0.761 |
| | | 400 | | | | | | |
| 18 | Sulfuric | PEG | — | 0.425 | — | 0.094 | 0.218 | 0.736 |
| | | 400 | | | | | | |
| 19 | Sulfurous | PEG | — | 0.410 | — | 0.069 | 0.239 | 0.719 |
| | | 400 | | | | | | |
| 20 | Citric | PEG | — | 0.424 | — | 0.084 | 0.282 | 0.790 |
| | | 400 | | | | | | |
| 21 | MSA | PEG | — | — | — | — | — | 0.000 |
| | | 200 | | | | | | |
| 22 | MSA | PEG | — | 0.269 | — | — | — | 0.269 |
| | | 300 | | | | | | |
| 23 | MSA | PEG | — | 0.432 | — | 0.093 | 0.264 | 0.789 |
| | | 600 | | | | | | |
| | | | Impurities, 2W | | | | | |
| Sample | | | Other | | | | | |
| ID | Aqueous Acids | PEGx | Impurities | g-TTX | o-TTX | e-TTX | a-TTX | Sum |
| 14 | Benzenesulfonic | PEG | — | 0.397 | 0.104 | 0.229 | 4.619 | 5.349 |
| | | 400 | | | | | | |
| 15 | Formic | PEG | — | 0.332 | 0.296 | 0.181 | 5.530 | 6.340 |
| | | 400 | | | | | | |
| 16 | HCl | PEG | — | 0.372 | 0.087 | 0.232 | 4.479 | 5.170 |
| | | 400 | | | | | | |
| 17 | Phosphoric | PEG | — | 0.347 | 0.286 | 0.240 | 5.640 | 6.513 |
| | | 400 | | | | | | |
| 18 | Sulfuric | PEG | — | 0.919 | — | 0.263 | 6.004 | 7.186 |
| | | 400 | | | | | | |
| 19 | Sulfurous | PEG | — | 0.342 | 0.125 | 0.319 | 5.192 | 5.978 |
| | | 400 | | | | | | |
| 20 | Citric | PEG | — | 0.329 | 0.869 | 0.180 | 6.987 | 8.365 |
| | | 400 | | | | | | |
| 21 | MSA | PEG | — | — | — | — | — | 0.000 |
| | | 200 | | | | | | |
| 22 | MSA | PEG | — | 0.305 | — | — | — | 0.305 |
| | | 300 | | | | | | |
| 23 | MSA | PEG | — | 0.361 | 0.141 | 0.392 | 4.846 | 5.740 |
| | | 600 | | | | | | |
| | | | Impurities, 4W | | | | | |
| Sample | | | Other | | | | | |
| ID | Aqueous Acids | PEGx | Impurities | g-TTX | 0-TTX | e-TTX | a-TTX | Sum |
| 14 | Benzenesulfonic | PEG | 0.799 | 0.404 | 0.478 | 0.541 | 9.521 | 11.74 |
| | | 400 | | | | | | |
| 15 | Formic | PEG | 0.875 | 0.383 | 0.908 | 0.489 | 11.187 | 13.84 |
| | | 400 | | | | | | |
| 16 | HCl | PEG | — | 0.390 | 0.241 | 0.415 | 8.980 | 10.03 |
| | | 400 | | | | | | |
| 17 | Phosphoric | PEG | 0.695 | 0.397 | 0.832 | 0.471 | 11.167 | 13.56 |
| | | 400 | | | | | | |
| 18 | Sulfuric | PEG | — | 0.425 | 0.312 | 0.530 | 10.368 | 11.63 |
| | | 400 | | | | | | |
| 19 | Sulfurous | PEG | — | 0.392 | 0.429 | 0.588 | 10.686 | 12.09 |
| | | 400 | | | | | | |
| 20 | Citric | PEG | — | 0.375 | 1.705 | 0.770 | 23.168 | 26.02 |
| | | 400 | | | | | | |
| 21 | MSA | PEG | — | — | — | — | — | 0 |
| | | 200 | | | | | | |
| 22 | MSA | PEG | — | 0.378 | — | — | — | 0.378 |
| | | 300 | | | | | | |
| 23 | MSA | PEG | 1.962 | 0.410 | 0.408 | 0.799 | 10.066 | 13.65 |
| | | 600 | | | | | | |

The results of this additional study were generally consistent with the original study. Strong acids were found to have a similar stabilizing effect for TTX, indicating the effect of the pH on the formulation. The study also confirms the advantage of using of PEG 400 in the formulation.

### Example 11—Secondary Percentage H₂O Study

A further study of the effect of the concentration of H₂O in the formulation was carried out on formulations comprising the combinations of aqueous MSA, PG and PEG. The formulations tested are outlined in Table 25. The stability is assessed at the temperature and times points noted in the last column. For the formulation shown in the last row of table 25 comprising 2% MSA, the amount of TTX may have been reduced slightly in cases where the TTX did not fully dissolve.

TABLE 25

| | | | PEG | | |
|---|---|---|---|---|---|
| TTX | MSA | PG | 400 | pH | Stability |
| 100 μg/mL | 50 | 26.3 | 23.7 | 5 | 40° C.: T0, 2 W, 4 W |
| 100 μg/mL | 25 | 13.2 | 11.8 | 5 | 40° C.: T0, 2 W, 4 W |
| 100 μg/mL | 10 | 47.3 | 42.7 | 5 | 40° C.: T0, 2 W, 4 W |
| 100 μg/mL | 5 | 50 | 45 | 5 | 40° C.: T0, 2 W, 4 W |
| 100 μg/mL | 2 | 51.5 | 46.5 | 5 | 40° C.: T0, 2 W, 4 W |
| (*may reduce | | | | | |
| to be soluble) | | | | | |

The study results are provided in Tables 26 and 27. Table 26 includes the measured pH values for each formulation at each time point and the percent of TTX relative to the TTX at T0 and Table 27 includes the relative percentage of the TTX degradation products (g-TTX, o-TTX, e-TTX, a-TTX) relative to the amount of these substances at T0.

TABLE 26

| | pH measured | | | Assay (mg/mL) | | | Assay (% LC) | | |
|---|---|---|---|---|---|---|---|---|---|
| % H2O | T0 | 2W | 4W | T0 | 2W | 4W | T0 | 2W | 4W |
| 50 | 4.34 | 3.73 | 3.69 | 0.1043 | 0.0883 | 0.0785 | 104.66% | 88.59% | 78.81% |
| 25 | 5.01 | 4.62 | 4.53 | 0.1013 | 0.0906 | 0.0815 | 101.93% | 91.12% | 82.00% |
| 10 | 5.24 | 5.11 | 5.02 | 0.1054 | 0.0971 | 0.0896 | 101.53% | 93.53% | 86.37% |
| 5 | 5.20 | 4.77 | 4.89 | 0.1013 | 0.0947 | 0.0887 | 100.06% | 93.54% | 87.64% |
| 2 | 5.22 | 5.14 | 5.23 | 0.1046 | 0.0981 | 0.0918 | 101.37% | 95.01% | 88.95% |

TABLE 27

| | Impurities, T0 | | | | | |
|---|---|---|---|---|---|---|
| % H2O | g-TTX | o-TTX | e-TTX | a-TTX | RRT = 2.5 | Sum |
| 50 | 0.442 | — | — | 0.209 | — | 0.651 |
| 25 | 0.442 | — | — | 0.255 | — | 0.697 |
| 10 | 0.416 | — | — | 0.205 | — | 0.620 |
| 5 | 0.444 | — | 0.109 | 0.212 | — | 0.766 |
| 2 | 0.437 | — | 0.072 | 0.202 | — | 0.711 |
| | Impurities, 2W | | | | | |
| % H2O | g-TTX | o-TTX | e-TTX | a-TTX | RRT = 2.5 | Sum |
| 50 | 0.246 | 0.317 | 1.501 | 10.255 | — | 12.320 |
| 25 | 0.374 | 0.210 | 0.893 | 7.930 | — | 9.407 |
| 10 | 0.384 | 0.150 | 0.488 | 6.294 | — | 7.315 |
| 5 | 0.356 | 0.140 | 0.298 | 5.420 | — | 6.215 |
| 2 | 0.370 | 0.189 | 0.301 | 5.235 | — | 6.095 |
| | Impurities, 4W | | | | | |
| % H2O | g-TTX | o-TTX | e-TTX | a-TTX | RRT = 2.5 | Sum |
| 50 | 0.329 | 0.755 | 3.116 | 16.986 | 0.941 | 21.19 |
| 25 | 0.351 | 0.510 | 1.827 | 13.730 | 1.075 | 16.42 |
| 10 | 0.354 | 0.320 | 0.911 | 11.036 | 1.132 | 12.62 |
| 5 | 0.343 | 0.259 | 0.564 | 9.749 | 0.940 | 10.92 |
| 2 | 0.400 | 0.387 | 0.566 | 9.310 | 1.035 | 10.66 |

From these results a clear trend emerges, that the higher the water content the higher the degree of TTX degradation.

### Example 12—Exemplary TTX Formulations

As would be understood from the present description, the liquid TTX formulations described herein are preferably in a form for parenteral administration. For example, the formulations described herein may be administered by I.M. or, preferably, S.C. routes to provide an initial dose of 30 μg of Tetrodotoxin to a patient. In another example, the liquid parenteral formulations described herein may also be administered to provide a daily dose of 60 μg of Tetrodotoxin to a patient. It will also be understood that the formulations described herein are also suitable for various other doses and dosage regimens as would be known to persons skilled in the art. In view of their enhanced stability profiles, the formulations described herein are well-suited for being stored for periods of time in containers, such as vials, ampules, syringes, auto injectors and the like. Thus, the formulations may be packaged in predetermined dosage amounts for use by a subject or for administration to a subject. In this regard, the formulation may, for example be packaged into pre-filled syringes or the like.

The following examples are provided to illustrate some possible TTX formulations of the present description.

### Example 12a

| Ingredients | Quantity | |
|---|---|---|
| Tetrodotoxin | 150 | μg |
| Water for injection | 0.05 | mL |
| Propylene glycol | 0.5 | mL |
| Polyethylene glycol 400 | 0.45 | mL |

| | |
|---|---|
| Acetic acid | Adjust pH to 4.0 ± 0.5 |

To a processing vessel, water for injection and acetic acid were added to obtain a solution with pH 2.0. Tetrodotoxin was added to the above solution and mixed until dissolved. Propylene glycol and polyethylene glycol 400 were added and mixed well. The pH of the solution was adjusted to 4.0±0.5 by addition of acetic acid if necessary. The solution was filtered and filled into containers.

### Example 12b

| Ingredients | Quantity | |
|---|---|---|
| Tetrodotoxin | 150 | μg |
| Water for injection | 0.05 | mL |
| Dimethylacetamide | 0.25 | mL |
| Polyethylene glycol 400 | 0.7 | mL |

| | |
|---|---|
| Methanesulfonic acid | Adjust pH to 4.0 ± 0.5 |

To a processing vessel, water for injection and methanesulfonic acid were added to obtain a solution with pH 2.0. Tetrodotoxin was added to the above solution and mixed until dissolved. Dimethylacetamide and polyethylene glycol 400 were added and mixed well. The pH of the solution was adjusted to 4.0±0.5 by addition of methanesulfonic acid if necessary. The solution was filtered and filled into containers.

### Example 12c

| Ingredients | Quantity | |
|---|---|---|
| Tetrodotoxin | 150 | μg |
| Water for injection | 0.05 | mL |
| Propylene glycol | 0.5 | mL |
| Polyethylene glycol 400 | 0.45 | mL |

| | |
|---|---|
| Methanesulfonic acid | Adjust pH to 4.0 ± 0.5 |

To a processing vessel, water for injection and methanesulfonic acid were added to obtain a solution with pH 2.0. Tetrodotoxin was added to the above solution and mixed until dissolved. Propylene glycol and polyethylene glycol 400 were added and mixed well. The PH of the solution was adjusted to 4.0±0.5 by addition of methanesulfonic acid if necessary. The solution was filtered and filled into containers.

### Example 12d

| Ingredients | Quantity | |
|---|---|---|
| Tetrodotoxin | 60 | μg |
| Water for injection | 0.02 | mL |
| Propylene glycol | 0.5 | mL |
| Polyethylene glycol 400 | 0.45 | mL |
| Sodium Acetate | 2.624 | mg |
| Acetic acid | 0.12 | mg |

| | |
|---|---|
| Methanesulfonic acid | Adjust pH to 6.0 ± 0.5 |

To a processing vessel, water for injection, sodium acetate and acetic acid were added to prepare a buffer solution. Tetrodotoxin was added to the above solution and mixed until dissolved. Propylene glycol and polyethylene glycol 400 were added and mixed well. The pH of the solution was adjusted to 6.0±0.5 by addition of methanesulfonic acid if necessary. The solution was filtered and filled into containers.

### Example 12e

| Ingredients | Quantity | |
|---|---|---|
| Tetrodotoxin | 60 | μg |
| Water for injection | 0.02 | mL |
| Propylene glycol | 0.5 | mL |
| Polyethylene glycol 400 | 0.45 | mL |
| Sodium citrate | 0.9804 | mg |
| Citric acid | 1.1904 | mg |

| | |
|---|---|
| Methanesulfonic acid | Adjust pH to 4.0 ± 0.5 |

To a processing vessel, water for injection, sodium citrate and citric acid were added to prepare a buffer solution. Tetrodotoxin was added to the above solution and mixed until dissolved. Propylene glycol and polyethylene glycol 400 were added and mixed well. The pH of the solution was adjusted to 4.0±0.5 by addition of methanesulfonic acid if necessary. The solution was filtered and filled into containers.

### Example 12f

| Ingredients | Quantity | |
|---|---|---|
| Tetrodotoxin | 60 | μg |
| Water for injection | 0.02 | mL |
| Propylene glycol | 0.5 | mL |
| Polyethylene glycol 400 | 0.45 | mL |
| Sodium citrate | 2.2446 | mg |
| Citric acid | 0.2496 | mg |

| | |
|---|---|
| Methanesulfonic acid | Adjust pH to 6.0 ± 0.5 |

To a processing vessel, water for injection, sodium citrate and citric acid were added to prepare a buffer solution. Tetrodotoxin was added to the above solution and mixed until dissolved. Propylene glycol and polyethylene glycol 400 were added and mixed well. The pH of the solution was adjusted to 4.0±0.5 by addition of methanesulfonic acid if necessary. The solution was filtered and filled into containers.

### Example 12g

| Ingredients | Quantity | |
|---|---|---|
| Tetrodotoxin | 60 | μg |
| Water for injection | 0.02 | mL |
| Propylene glycol | 0.5 | mL |
| Polyethylene glycol 400 | 0.45 | mL |
| Sodium phosphate, monobasic | 2.4 | mg |
| Phosphoric acid | 0.0294 | mg |

| | |
|---|---|
| Methanesulfonic acid | Adjust pH to 4.0 ± 0.5 |

To a processing vessel, water for injection, sodium phosphate monobasic and phosphoric acid were added to prepare a buffer solution. Tetrodotoxin was added to the above solution and mixed until dissolved. Propylene glycol and polyethylene glycol 400 were added and mixed well. The pH of the solution was adjusted to 4.0±0.5 by addition of methanesulfonic acid if necessary. The solution was filtered and filled into containers.

### Example 12h

| Ingredients | Quantity | |
|---|---|---|
| Tetrodotoxin | 60 | μg |
| Water for injection | 0.02 | mL |
| Propylene glycol | 0.5 | mL |
| Polyethylene glycol 400 | 0.45 | mL |
| Sodium phosphate, monobasic | 1.8 | mg |
| Sodium phosphate, dibasic | 0.142 | mg |

| | |
|---|---|
| Methanesulfonic acid | Adjust pH to 6.0 ± 0.5 |

To a processing vessel, water for injection, sodium phosphate monobasic and sodium phosphate dibasic were added to prepare a buffer solution. Tetrodotoxin was added to the above solution and mixed until dissolved. Propylene glycol and polyethylene glycol 400 were added and mixed well. The pH of the solution was adjusted to 6.0±0.5 by addition of methanesulfonic acid if necessary. The solution was filtered and filled into containers.

### Example 13—Stability of TTX Formulations Stored in Pre-Filled Syringes

A study was conducted to determine the stability characteristics of TTX formulations stored in pre-filled syringes. A TTX formulation comprising methanesulfonic acid (MSA), propylene glycol (PG) and polyethylene glycol (PEG) was prepared as described above. The samples were filled into BD Neopak 1 ml 27 g glass syringes. The stability of the samples was assessed under the following conditions and time periods as set out in Table 28:

TABLE 28
Stability conditions

| 40° C./75% RH | 25° C./40% RH | 2-8° C. |
|---|---|---|
| T0 for all samples | T0 for all samples | T0 for all samples |
| 2 w | | |
| 4 w | 1 m | 1 m |
| | 3 m | 3 m |
| | | 6 m |
| | | 12 m |

The relative amount of TTX and TTX impurities was determined for each of the above conditions and time periods as shown in Table 29. The determination of the relative amounts of the impurities was done by HPLC in comparison to sample at time 0 (T0) by the method described above.

TABLE 29

| | | | Assay | Assay | Impurities (%) | | | | | |
|---|---|---|---|---|---|---|---|---|---|---|
| Conditions | Analysis | pH | (mg/mL) | (% LC) | g-TTX | o-TTX | e-TTX | a-TTX | RRT = 1.053 | Sum |
| | T0 | 5.08 | 0.0987 | 101.0% | 0.572 | 0.445 | 0.167 | 0.527 | 0.420 | 2.214 |
| 40° C./75% | 2w | 5.15* | 0.0935 | 95.66% | 0.632 | 0.450 | 0.305 | 5.482 | 0.272 | 7.141 |
| RH | | | | | | | | | | |
| 40° C./75% | 4w | 6.46 | 0.0886 | 90.6% | 0.551 | 0.454 | 0.468 | 10.421 | 0.292 | 12.186 |
| RH | | | | | | | | | | |
| 25° C./40% | 1M | 5.77 | 0.0987 | 101.0% | 0.573 | 0.352 | 0.167 | 1.560 | 0.374 | 3.028 |
| RH | | | | | | | | | | |
| 25° C./40% | 3M | 5.54 | 0.0983 | 100.6% | 0.587 | 0.377 | 0.133 | 2.651 | 0.421 | 4.169 |
| RH | | | | | | | | | | |
| 2-8° C. | 1M | 6.01 | 0.0996 | 102.0% | 0.634 | 0.360 | 0.171 | 0.606 | 0.331 | 2.103 |
| 2-8° C. | 3M | 5.35 | 0.0984 | 100.7% | 0.672 | 0.355 | 0.191 | 0.808 | 0.416 | 2.441 |
| 2-8° C. | 6M | 6.00 to | 0.0981 | 100.4% | 0.610 | 0.531 | 0.239 | 1.078 | Not observed² | 2.458 |
| | | 6.92¹ | | | | | | | | |
| 2-8° C. | 12M | 6.36 | 0.0973 | 99.5% | 0.761 | 0.575 | 0.288 | 1.414 | 0.275 | 3.313 |

*Initial observed value was 5.79, however additional stirring due to viscosity of sample helped stabilizing the pH reading.
¹A first pH reading of 6.92 was obtained using a pooled solution from 2 of the 4 syringes kept for pH analysis at 2-8° C. The analysis of the same solution on another day yielded a pH of 6.74. Pooling the remaining two syringes in a new solution for pH analysis yielded pH values of 6.00 and 6.14.
²The peak/hump in the tailing of the TTX peak (RRT ≈ 1.045) was not observed.

### Example 14—Order of Addition Study

To investigate the potential impact of the order of addition of the components on TTX solubility, as series of experiments were run altering the order of addition of the components of the composition. First, a TTX formulation having the following excipient ratios PG:PEG 400:MSA (50:45:5) (where MSA is 0.01M aqueous MSA) was prepared as a standard preparation. The pH and appearance were recorded after each step. The results of this experiment are reported in Table 30. In Experiments A to G, TTX formulations having the same composition as the standard preparation were prepared but the order of the addition steps was altered. The pH and appearance were recorded after each addition step. The results of Experiments A to G are shown in Tables 31-37 which include the order of addition of the components and the pH and appearance at each step. A stock solution of TTX in MSA/H₂O was prepared and used in the Experiments B-G shown in Tables 32-37.

TABLE 30

| Sample | Steps | Observations |
|---|---|---|
| Standard | Add MSA in H₂O | pH = 2.11 |
| | | Appearance: Clear solution |
| Preparation | Add TTX to | pH = 2.58 |
| | MSA/H₂O | Solubilization time: 30 s |
| | | Appearance: Clear solution |
| | Add PG to | pH = 2.98 |
| | TTX/MSA/H₂O | Appearance: Clear solution after |
| | | gently mixing/swirling. |
| | Add PEG 400 to | pH = 5.10 |
| | TTX/MSA/H₂O/PG | Appearance: Clear solution after |
| | | gently mixing/swirling. |

TABLE 31

| Sample | Steps | Observations |
|---|---|---|
| Experiment A | Add TTX in H₂O | pH = 8.95 |
| | | Solubilization time: Not fully |
| | | dissolved after mixing >2 min. |
| | | Appearance: Cloudy solution. |
| | Add MSA to TTX/ | pH = 2.56 |
| | H₂O | Appearance: Clear solution after |
| | | swirling 10 s. |
| | Add PG to | pH = 2.91 |
| | TTX/MSA/H₂O | Appearance: Clear solution after |
| | | gently mixing/swirling. |
| | Add PEG 400 to | pH = 4.89 |
| | TTX/MSA/H₂O/PG | Appearance: Clear solution after |
| | | gently mixing/swirling. |

TABLE 32

| Sample | Steps | Observations |
|---|---|---|
| Experiment B | Add TTX Stock into | pH = 2.95 |
| | PG | Appearance: Clear solution after |
| | | gently mixing/swirling. |
| | Add PEG 400 | pH = 5.01 |
| | | Appearance: Clear solution after |
| | | gently mixing/swirling. |

TABLE 33

| Sample | Steps | Observations |
|---|---|---|
| Experiment C | Add PEG 400 into | pH = 5.59 |
| | TTX Stock | Appearance: Clear solution after |
| | | gently mixing/swirling. |
| | Add PG | pH = 5.24 |
| | | Appearance: Clear solution after |
| | | gently mixing/swirling. |

TABLE 34

| Sample | Steps | Observations |
|---|---|---|
| Experiment D | Add TTX Stock | pH = 4.91 |
| | into PEG 400 | Appearance: Clear solution after |
| | | gently mixing/swirling. |
| | Add PG | pH = 5.18 |
| | | Appearance: Clear solution after |
| | | gently mixing/swirling. |

TABLE 35

| Sample | Steps | Observations |
|---|---|---|
| Experiment E | PG:PEG400 | pH = 5.93 |
| | (53:47) | Appearance: Clear solution. |
| | Add TTX Stock | pH = 5.06 |
| | to PG:PEG400 | Appearance: Clear solution after |
| | (53:47) | gently mixing/swirling. |

TABLE 36

| Sample | Steps | Observations |
|---|---|---|
| Experiment F | TTX Stock in | pH = 2.67 |
| | MSA 0.01M | Appearance: Clear solution. |
| | Add PG:PEG400 | pH = 4.90 |
| | (53:47) to TX Stock | Appearance: Clear solution after |
| | | gently mixing/swirling. |

TABLE 37

| Sample | Steps | Observations |
|---|---|---|
| Experiment G | Add PG into | pH = 2.88 |
| | TTX Stock | Appearance: Clear solution after |
| | | gently mixing/swirling. |
| | Add PEG 400 | pH = 4.94 |
| | | Appearance: Clear solution after |
| | | gently mixing/swirling. |

The TTX formulations prepared by the standard procedure and in the Experiments A-G (Tables 30-37) were assayed to determine the amount of TTX and the amount of impurities obtained by each process. The assay and impurity test results are shown in Table 38 below. The percentages are w/v.

TABLE 38

| | TTX | g-TTX | o-TTX | e-TTX | a-TTX | Sum |
|---|---|---|---|---|---|---|
| Samples | (%) | (%) | (%) | (%) | (%) | (%) |
| Standard | 95.9 | 0.46 | 0.36 | 0.18 | 0.50 | 1.50 |
| Preparation | | | | | | |
| Experiment A | 95.6 | 0.47 | 0.37 | 0.18 | 0.51 | 1.53 |
| Experiment B | 96.6 | 0.45 | 0.35 | 0.15 | 0.54 | 1.49 |
| Experiment C | 96.6 | 0.44 | 0.28 | 0.19 | 0.54 | 1.45 |
| Experiment D | 96.5 | 0.43 | 0.32 | 0.21 | 0.52 | 1.48 |
| Experiment E | 96.8 | 0.46 | 0.32 | 0.14 | 0.55 | 1.48 |
| Experiment F | 96.4 | 0.45 | 0.31 | 0.12 | 0.58 | 1.45 |
| Experiment G | 96.7 | 0.43 | 0.31 | 0.18 | 0.55 | 1.47 |

### C) Efficacy Study

### Example 15—Efficacy of TTX Formulations in Rats with Chemotherapy-Induced Neuropathy

A study comparing the efficacy of TTX lyophilized formulation and TTX liquid formulation in the treatment of rats with chemotherapy induced neuropathy was undertaken. For this study Adult male Sprague-Dawley rats were used. Chemotherapy-induced peripheral neuropathy (CIPN) was induced by oxaliplatin 4 mg/kg, iv, administered twice a week for up to 3 weeks. Induction of neuropathy was confirmed by assessment of mechanical allodynia using von Frey hairs (PWT≤4 g). Rats with induced mechanical allodynia were divided into 7 groups, with relevant treatments for 5 days. Details are set out in table 39 below. Paw withdrawal threshold (PWT) was tested daily for three days before beginning the treatment course (pre test). daily before dosing, 1 hour after dosing and 2 hours after dosing during the 5 day treatment course, and once on the 7th, 10th and 14th days after the start of the dosing course. PWT was expressed as mean±SEM. One-way ANOVA was used to compare different groups at the same time points.

TABLE 39

| | | Dose | Dose | | Dose | | | | |
|---|---|---|---|---|---|---|---|---|---|
| Group | Description | level | volume | | concentration | | Schedule | Route | n |
| 1 | Normal saline, sterile | 0 μg/kg | 1 | ml/kg | 0 | μg/mL | qd for 5 d | SC | 7 |
| 2 | Lyophilized TTX for | 8 μg/kg | 1 | ml/kg | 8 | μg/mL | qd for 5 d | SC | 7 |
| | injection (Lyo TTX | | | | | | | | |
| | 8 μpk) | | | | | | | | |
| 3 | Stable liquid TTX | 10 μg/kg | 1.87 | mL/kg | 5.35 | μg/mL | qd for 5 d | SC | 7 |
| | formulation (SL TTX10 | | | | | | | | |
| | μpk) | | | | | | | | |
| 4 | Stable liquid TTX | 8 μg/kg | 1.86 | mL/kg | 4.3 | μg/mL | qd for 5 d | SC | 7 |
| | formulation (SL TTX8 | | | | | | | | |
| | μpk) | | | | | | | | |
| 5 | Vehicle for liquid | 0 μg/kg | 2 | mL/kg | 0 | μg/mL | qd for 5 d | SC | 7 |
| | formulation (SL | | | | | | | | |
| | vehicle) | | | | | | | | |
| 6 | Duloxetine (DXT | 30 mg/kg | 2 | mL/kg | 15 | mg/mL | qd for 5 d | PO | 7 |
| | 30 mpk, qd) | | | | | | | | |
| 7 | Pregabalin (PGN | 30 mg/kg | 2 | mL/kg | 15 | mg/mL | bid for 5 d | PO | 7 |
| | 30 mpk, bid) | | | | | | | | |

For this study the lyophilized TTX formulation comprises lyophilized TTX reconstituted in water then diluted to a concentration of 8 μg/mL and the stable liquid formulation is formulation of TTX having an excipient ratio of PG:PEG 400:MSA (50:45:5) (where MSA is 0.01M aqueous MSA) prepared by the Standard Preparation method described in example 14. The lyophilized TTX was administered at a dose of 8 μg/kg while the stable liquid formulation was tested at doses of 8 μg/kg and 10 μg/kg.

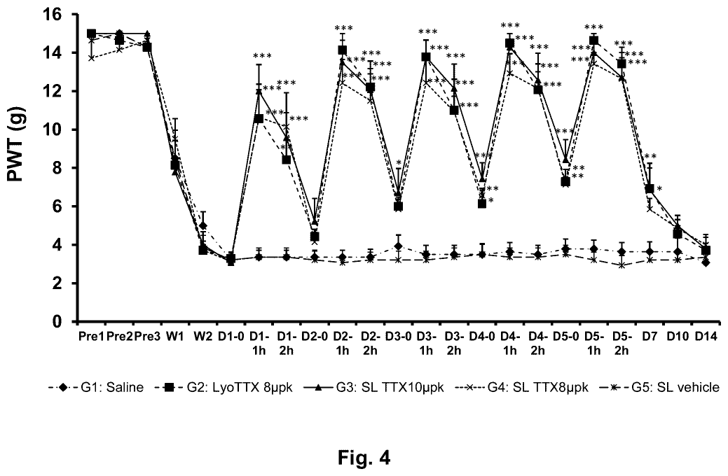

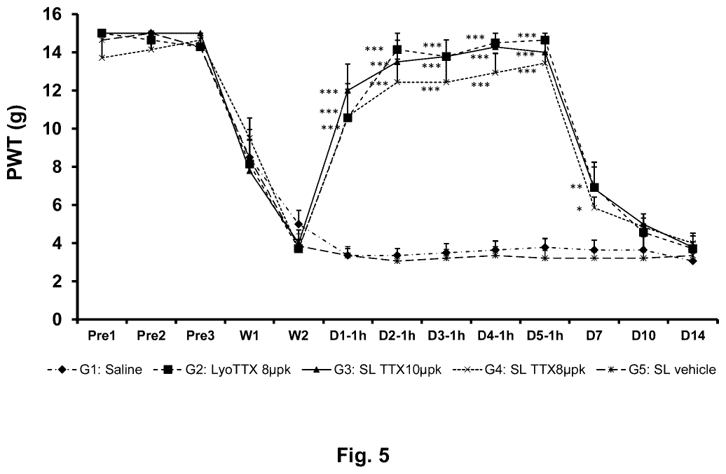

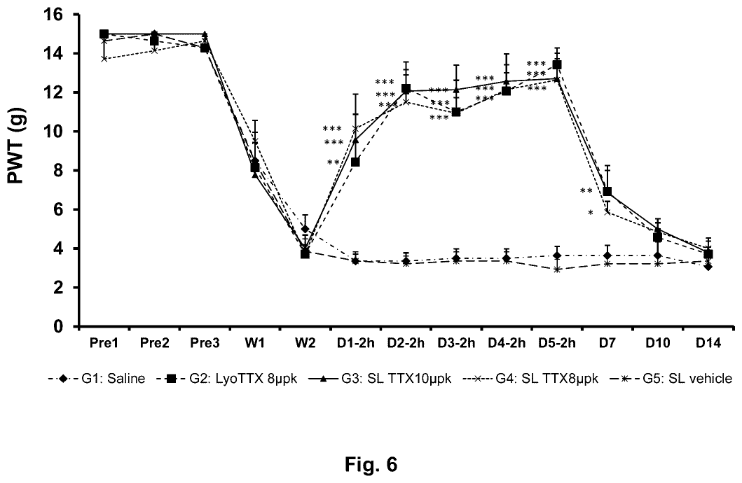

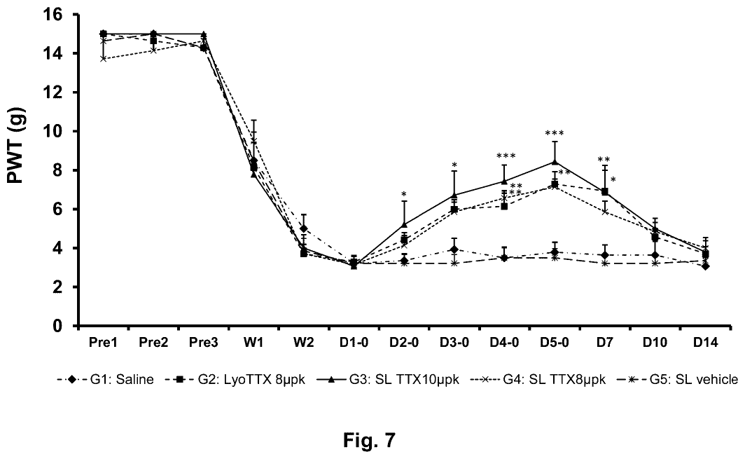

The results of the mechanical allodynia assessment for the two TTX formulations is shown in FIGS. 4-7. Normal saline and the vehicle of the liquid formulation were used as controls samples. FIG. 4 illustrates the PWT test results at all testing time points. The testing time points include daily testing for 3 days before oxaliplatin administration (Pre1, Pre2, Pre3), weekly testing for two weeks after oxaliplatin administration (W1, W2), testing 3 times per day during the TTX dosing course (D-0, before TTX dosing, D-1h, one hour post TTX dosing, and D-2h, two hours post TTX dosing), and once a day testing on days 7, 10 and 14 post TTX dosing. In order to appreciate the effects of TTX at the different time points during dosing, Graphs 5-7 isolate the data for each of the 1 hour, 2 hour and immediate before TTX dosing, time periods, so that comparisons can be more readily observed.

From these results it was found that no significant difference was observed on baseline and post-dosing PWT between CINP rats treated with normal saline or vehicle for liquid TTX formulation. This indicates that the vehicle is not a significant factor in the PWT results. Lyophilized TTX for injection at 8 μg/kg, sc, once daily, and Stable Liquid TTX formulation at 8 μg/kg, and 10 μg/kg sc, once daily all significantly increased baseline and post-dosing PWT in CINP rats which is demonstrated over FIGS. 4-7. However, there did not appear to be a significant difference in the efficacy of the different TTX formulations as the PWT test results of lyophilized and liquid formulations of TTX appear to overlap within the margin of error. This experiment demonstrates the utility of TTX liquid formulation.

Although the above description includes reference to certain specific embodiments, various modifications thereof will be apparent to those skilled in the art. Any examples provided herein are included solely for the purpose of illustration and are not intended to be limiting in any way. Any drawings provided herein are solely for the purpose of illustrating various aspects of the description and are not intended to be drawn to scale or to be limiting in any way. The scope of the claims appended hereto should not be limited by the preferred embodiments set forth in the above description but should be given the broadest interpretation consistent with the present specification as a whole. The disclosures of all prior art recited herein are incorporated herein by reference in their entirety.

1. A stable liquid formulation comprising tetrodotoxin, and/or a derivative, analog, or a pharmaceutically acceptable salt thereof, and one or more pharmaceutically acceptable diluents, carriers, and excipients wherein the formulation comprises an aqueous component and a non-aqueous organic component and wherein the aqueous component comprises about 2% to about 10% (v/v) of the formulation.

2. The formulation of claim 1, wherein the formulation further comprises one or more: solvents; pH adjusting agents; buffering agents; and stabilizing agents.

3. The formulation of claim 1, wherein the tetrodotoxin and/or a derivative, analog, or a pharmaceutically acceptable salt thereof, is in the formulation at a concentration between about 5 and about 5000 μg/mL.

4. (canceled)

5. (canceled)

6. The formulation of claim 1, wherein the aqueous component is present from about 2% to about 5% (v/v) of the formulation.

7. The formulation of claim 1, wherein the organic component is present from about 90% to about 98% (v/v) of the formulation.

8. (canceled)

9. The formulation of claim 1, wherein the aqueous component comprises an aqueous acid solution, wherein the aqueous acid solution comprises one or more of hydrochloric acid, acetic acid, anhydrous citric acid, benzenesulfonic acid, citric acid monohydrate, lactic acid, (DL)-lactic acid, (L)-lactic acid, methanesulfonic acid, and phosphoric acid.

10. (canceled)

11. (canceled)

12. The formulation of claim 1, wherein the organic component comprises one or more of ethyl alcohol, dehydrated ethyl alcohol, denatured ethyl alcohol, benzyl alcohol, dimethyl sulfoxide, glycerin, isopropyl alcohol, methylpyrrolidone, N,N-dimethylacetamide, polyethylene glycol, and propylene glycol.

13. The formulation of claim 12, wherein the organic component is combination of polyethylene glycol and propylene glycol, wherein the polyethylene glycol comprises polyethylene glycol 200 (PEG 200), polyethylene glycol 300 (PEG 300), polyethylene glycol 400 (PEG 400), and/or polyethylene glycol 600 (PEG 600).

14. (canceled)

15. The formulation of claim 12, wherein the formulation comprises an aqueous acid, polyethylene glycol, and propylene glycol in a ratio of between about 5:80:15 to 5:20:75 (v/v).

16. (canceled)

17. (canceled)

18. The formulation of claim 15, wherein the acid is MSA and wherein the PEG is PEG 400.

19. (canceled)

20. The formulation of claim 1, wherein the pH of the formulation is from about 3 to about 6.

21. (canceled)

22. The formulation of claim 20, wherein the pH of the formulation is adjusted with hydrochloric acid, acetic acid, acetic anhydride, adipic acid, anhydrous citric acid, benzenesulfonic acid, boric acid, citric acid monohydrate, lactic acid, (DL)-lactic acid, (L)-lactic acid, maleic acid, metaphosphoric acid, methanesulfonic acid, nitric acid, phosphoric acid, succinic acid, sulfuric acid, sulfurous acid, tartaric acid, (DL)-tartaric acid, trifluoroacetic acid, ascorbic acid, benzoic acid, edetic acid, formic acid, lactobionic acid, aspartic acid, caprylic acid, glucuronic acid, hydroxyethylpiperazine ethane sulfonic acid, methylboronic acid, oleic acid, palmitic acid, pentetic acid, stearic acid, sodium hydroxide, calcium hydroxide, potassium hydroxide, sodium bicarbonate, sodium carbonate, sodium carbonate decahydrate, sodium carbonate monohydrate, diethanolamine, meglumine, tromethamine, and/or ammonia.

23. The formulation of claim 1, wherein the formulation is adapted to provide a dose of about 5 to about 120 μg, about 15 to about 60 μg, or about 30 μg of tetrodotoxin, and/or a derivative, analog, or a pharmaceutically acceptable salt thereof.

24. The formulation of claim 23, wherein the formulation is provided in a container, wherein the container is a vial, an ampule, or a syringe.

25. (canceled)

26. The formulation of claim 1, wherein the formulation is adapted for intramuscular (IM) or subcutaneous (SC) administration.

27. The formulation of claim 1, wherein less than 10% of the tetrodotoxin, and/or a derivative, analog, or a pharmaceutically acceptable salt thereof, undergoes degradation after being stored at 40° C. for a period of 28 days.

28. The formulation of claim 1, wherein less than 10% of the tetrodotoxin, and/or a derivative, analog, or a pharmaceutically acceptable salt thereof, undergoes degradation after being stored at 25° C. for a period of 4 weeks.

29. The formulation of claim 1, wherein less than 10% of the tetrodotoxin, and/or a derivative, analog, or a pharmaceutically acceptable salt thereof, undergoes degradation after being stored at a temperature between 2-8° C. for a period of 3 months.

30. A pre-filled syringe comprising a predetermined volume of the formulation according to claim 1.

31. (canceled)

32. (canceled)

33. (canceled)

34. (canceled)

35. A method of treating or preventing chemotherapy induced peripheral neuropathy in a subject in need thereof comprising administering to the subject a stable liquid formulation according to claim 1.

36. (canceled)
